(19) United States
(12) Patent Application Publication
(10) Pub. No.: US 2023/0165715 A1
(43) Pub. Date: Jun. 1, 2023

(54) INTRAOCULAR CYCLOPHOTOCOAGULATION DEVICE AND METHODS OF USE

(71) Applicant: Innovation Drive Corporation, Reno, NV (US)

(72) Inventors: Matthew Schuster, Reno, NV (US); Luke W. Clauson, Reno, NV (US); Nicholas G. Lewis, Reno, NV (US); Matthew B. Newell, Reno, NV (US)

(21) Appl. No.: 18/072,389

(22) Filed: Nov. 30, 2022

Related U.S. Application Data
(60) Provisional application No. 63/264,703, filed on Nov. 30, 2021.

(51) Int. Cl. A61F 9/008

(52) U.S. Cl. CPC A61F 9/00823; A61F 2009/00861

(57) ABSTRACT

An intraocular cyclophotocoagulation device having a proximal, reusable portion and a distal, disposable portion. The reusable portion includes laser treatment assembly comprising a laser diode and a collimating lens positioned a distance distal to the laser diode and configured to collimate light from the laser diode into a collimated laser beam and direct the collimated laser beam towards a distal end region of the proximal housing portion; and an imaging assembly. The disposable portion includes a laser guide extending through an elongate shaft and an aspheric lens positioned within the distal housing portion to receive the collimated laser beam from the proximal reusable portion and direct the collimated laser beam toward a proximal end of a fiberoptic of the laser guide and an illumination light guide and an illumination source positioned within the distal housing portion. Related devices, systems, and methods are provided.

### CROSS-REFERENCE TO RELATED APPLICATIONS

This application claims the benefit of priority under 35 U.S.C. § 119(e) to co-pending U.S. Provisional Patent Application Ser. No. 63/264,703, filed Nov. 30, 2021. The disclosure of the application is incorporated by reference in its entirety.

### BACKGROUND

Glaucoma is a complicated disease in which damage to the optic nerve leads to progressive vision loss and is the leading cause of irreversible blindness. Aqueous humor is the fluid that fills the anterior chamber in front of the iris and the posterior chamber of the eye behind the iris. Vitreous humor or vitreous body is a gel-like material found in the posterior segment of the eye posterior of the capsular bag. FIG. 1 is a diagram of the front portion of an eye 5 showing the lens 7, cornea 8, iris 9, ciliary body 6 including ciliary processes 4, trabecular meshwork 10, and Schlemm's canal 12. The aqueous humor is a fluid produced by the ciliary body 6 that lies behind the iris 9 adjacent to the lens 7. This aqueous humor washes over the lens 7 and iris 9 and flows to the drainage system located in the angle of the anterior chamber. The angle of the anterior chamber, which extends circumferentially around the eye, contains structures that allow the aqueous humor to drain.

Some of the aqueous humor is absorbed through the trabecular meshwork 10 into Schlemm's canal 12 into collector channels and passing through the sclera 15 into the episcleral venous circulation. The trabecular meshwork 10 extends circumferentially around the anterior chamber 16 in the angle. The trabecular meshwork 10 limits the outflow of aqueous humor. Schlemm's canal 12 is located beyond the trabecular meshwork 10. The two arrows in the anterior chamber 16 of FIG. 1 show the flow of aqueous humor from the ciliary body 6, over the lens 7, over the iris 9, through the trabecular meshwork 10, and into Schlemm's canal 12 and its collector channels.

In some cases glaucoma is caused by blockage of aqueous humor outflow such as by sclerosis of the trabecular meshwork, pigment or membrane in the angle. In other cases, blockage is due to a closure of the angle between the iris and the cornea. This angle type of glaucoma is referred to as “angle-closure glaucoma”. In the majority of glaucoma cases, however, called “open angle glaucoma”, the cause is unknown.

Treatments of glaucoma attempt to lower intraocular pressure (TOP) pharmacologically or by surgical intervention that enhance outflow of aqueous humor through the outflow pathways. Ab externo trabeculectomy is a type of glaucoma surgery that creates a new path as a “controlled” leak for fluid inside the eye to drain out. Conventionally, a partial thickness scleral flap is formed followed by the creation of a small hole into the anterior chamber. Aqueous humor can flow into the subconjunctival space creating a filtering bleb. The scleral flap is raised up and a blade used to enter the anterior chamber. During the operation a hole is created under the scleral flap that is fluidically connected to the anterior chamber creating an opening. The opening is partially covered with the scleral flap. A small conjunctival “bleb” or bubble appears over the scleral flap, often near the junction of the cornea and the sclera (limbus).

Minimally-invasive surgical procedures provide TOP lowering by enhancing the natural drainage pathways of the eye with minimal tissue disruption. Minimally-invasive glaucoma surgery (MIGS) uses microscopic-sized equipment and tiny incisions. MIGS offers an alternative to conventional glaucoma surgeries with the potential benefit of reducing a patient's dependence on topical glaucoma medication. Trabeculectomies and trabeculotomies can each be performed ab interno, or from inside the anterior chamber. Ab interno approaches aim to decrease TOP by increasing aqueous humor outflow through a direct opening in the trabecular meshwork from within the anterior chamber so that there is direct communication between the anterior chamber and the outer wall of Schlemm's canal. Ab interno approaches include the TRABECTOME (MST/NeoMedix Corp.) electrosurgical instrument that ablates and removes trabecular meshwork, the Kahook Dual Blade (New World Medical) for excisional goniotomy removing a strip of trabecular meshwork, gonioscopy assisted transluminal trabeculotomy (GATT) involving cutting through the trabecular meshwork, cannulating Schlemm's canal, and Omni (Sight Sciences) for performing viscoplasty or trabeculotomy through an ab interno approach for cannulating Schlemm's canal. Other ab interno methods include the iStent (Glaukos) to create pathway through the trabecular meshwork for improved outflow of aqueous humor through Schlemm's canal.

Cyclodestructive therapies are also known. Endoscopic cyclophotocoagulation uses lasers to decrease intraocular pressure by causing coagulative necrosis and shrinkage of the ciliary body, which reduces the production of aqueous in the eye.

Laser video endoscopes that would be used to perform cyclodestructive therapies are typically endoscopes that are reused after autoclaving or other sterilization method. Repeated sterilization tends to damage the optical elements of the endoscope over time. However, the endoscope components are generally too expensive to manufacture to justify disposing of them after a single use. Endoscopic systems also suffer from bulky and complicated assemblies that often provide low resolution imaging and visibility as well as inaccurate targeting of laser light that can lead to inadvertent tissue damage.

In view of the foregoing, there is a need for improved devices and methods related to ophthalmic surgery for the treatment of glaucoma.

### SUMMARY

In an aspect, described is an intraocular cyclophotocoagulation device. The device includes a proximal, reusable portion having a proximal housing portion having a distal end region; a laser treatment assembly having a laser diode and a collimating lens positioned a distance distal to the laser diode and configured to collimate light from the laser diode into a collimated laser beam and direct the collimated laser beam towards the distal end region of the proximal housing portion; and an imaging assembly. The device includes a distal, disposable portion having a distal housing portion with an elongate shaft extending distally from a distal end region of the distal housing portion, the distal housing portion having a proximal end region configured to reversibly couple with the distal end region of the proximal housing portion; a laser guide extending through the elongate shaft and an aspheric lens positioned within the distal housing portion to receive the collimated laser beam from the proximal reusable portion and direct the collimated laser beam toward a proximal end of a fiberoptic of the laser guide; an imaging guide extending through the elongate shaft and an objective lens located at a distal end region of the elongate shaft, the objective lens arranged to focus an image onto a distal end of a fiberoptic of the imaging guide; and an illumination light guide extending through the elongate shaft, an illumination source positioned within the distal housing portion and arranged relative to a proximal end of a fiberoptic of the illumination light guide.

The laser diode of the laser treatment assembly can transmit in a near-infrared wavelength that is configured to burn and shrink tissue. The collimated laser beam can be unchanged with minimal divergence or convergence crossing a junction from the reusable portion to the disposable portion. The aspheric lens can correct for spherical aberration and provide focus of both paraxial and marginal rays so that all light of the collimated laser beam enters the proximal end of the fiberoptic of the laser guide. The fiberoptic of the laser guide can be about 200 microns in diameter and the collimated laser beam can have a focusing spot that is about 100 microns. The objective lens can be monolithic and apertureless. The objective lens can have a frustoconical configuration having a smaller diameter entry surface and a maximal diameter exit surface, the exit surface positioned proximal to the entry surface near the distal end of the fiberoptic of the imaging guide. The objective lens can have an hourglass configuration with an entry surface, an exit surface and a neck located between the entry surface and the exit surface. The neck can have a smaller diameter than a diameter of the entry surface or a diameter of the exit surface. The objective lens can have a depth of focus that is between 1-6 mm. The proximal end of the fiberoptic of the illumination light guide can be affixed on or near an emitting die of the illumination source. The fiberoptic of the illumination light guide can be potted in a recess of the illumination source. The recess can have a curved bottom and together with an optical adhesive in the recess create a focusing lens between the fiberoptic of the illumination light guide and the emitting die of the illumination source.

The device can further include an actuator that is a slider configured to increase intensity of the laser light emitted. The slider can be positioned on the disposable portion. The elongate shaft can be curved. The device can provide a field of view that is between 45-150 degrees. The distal end region of the proximal housing portion can taper and be sized to be received within a corresponding shaped region at the proximal end region of the distal housing portion. The distal end region of the proximal housing portion and the proximal end region of the distal housing portion can couple together under a spring-load.

The device can further include a fluid channel extending within the disposable portion. The fluid channel can extend through the elongate shaft. The fluid channel is configured to deliver cooled liquid to an eye. The cooled liquid can be water or saline. The fluid channel is configured to deliver a therapeutic agent.

In an interrelated implementation, provided is an intraocular cyclophotocoagulation device having a proximal, reusable portion and a distal, disposable portion. The reusable portion includes a proximal housing portion having a distal end region; a laser treatment assembly having a laser diode and a collimating lens positioned a distance distal to the laser diode and configured to collimate light from the laser diode into a collimated laser beam and directed the collimated laser beam towards the distal end region of the proximal housing portion; and an imaging assembly. The distal, disposable portion includes a distal housing portion having an elongate shaft extending distally from a distal end region of the distal housing portion, the distal housing portion having a proximal end region configured to reversibly couple with the distal end region of the proximal housing portion; a laser guide extending through the elongate shaft and an aspheric lens positioned within the distal housing portion to receive the collimated laser beam from the proximal, reusable portion and direct the collimated laser beam toward a proximal end of a fiberoptic of the laser guide; an imaging guide extending through the elongate shaft and an apertureless, monolithic objective lens located at a distal end region of the elongate shaft, the objective lens arranged to focus an image onto a distal end of a fiberoptic of the imaging guide; and an illumination light guide extending through the elongate shaft to transmit light from an illumination source for illuminating an interior of the eye.

The laser diode of the laser treatment assembly can transmit in a near-infrared wavelength that is configured to burn and shrink tissue. The collimated laser beam can be unchanged with minimal divergence or convergence crossing a junction from the reusable portion to the disposable portion. The aspheric lens can correct for spherical aberration and provide focus of both paraxial and marginal rays so that all light of the collimated laser beam enters the proximal end of the fiberoptic of the laser guide. The fiberoptic of the laser guide can be about 200 microns in diameter and the collimated laser beam have a focusing spot that is about 100 microns. The objective lens can have a frustoconical configuration having a smaller diameter entry surface and a maximal diameter exit surface, the exit surface positioned proximal to the entry surface near the distal end of the fiberoptic of the imaging guide. The objective lens can have an hourglass configuration with an entry surface, an exit surface and a neck located between the entry surface and the exit surface, wherein the neck has a smaller diameter than a diameter of the entry surface or a diameter of the exit surface. The objective lens can have a depth of focus that is between 1-6 mm. The illumination light guide can include a fiberoptic and the illumination source can include an emitting die. The proximal end of the fiberoptic of the illumination light guide can be affixed on or near the emitting die of the illumination source. The fiberoptic of the illumination light guide can be potted in a recess of the illumination source. The recess can have a curved bottom and together with an optical adhesive in the recess create a focusing lens between the fiberoptic of the illumination light guide and the emitting die of the illumination source.

The device can further include an actuator that is a slider configured to increase intensity of the laser light emitted. The slider can be positioned on the disposable portion. The elongate shaft can be curved. The device can provide a field of view that is between 45-150 degrees. The distal end region of the proximal housing portion can taper and be sized to be received within a corresponding shaped region at the proximal end region of the distal housing portion. The distal end region of the proximal housing portion and the proximal end region of the distal housing portion couple under a spring-load.

The device can further include a fluid channel extending within the disposable portion. The fluid channel can extend through the elongate shaft. The fluid channel is configured to deliver cooled liquid to an eye. The cooled liquid can be water or saline. The fluid channel is configured to deliver a therapeutic agent.

In an interrelated implementation, provided is an intraocular cyclophotocoagulation device having a proximal, reusable portion having a proximal housing portion having a distal end region; and an imaging assembly having an image sensor and a lens element. The device includes a distal, disposable portion having a distal housing portion having an elongate shaft extending distally from a distal end region of the distal housing portion, the distal housing portion having a proximal end region configured to reversibly couple with the distal end region of the proximal housing portion; an imaging guide extending through the elongate shaft and an objective lens located at a distal end region of the elongate shaft distal to a distal end of the imaging guide, the objective lens arranged to focus an image onto the distal end of the imaging guide; an illumination light guide extending through the elongate shaft, an illumination source positioned within the distal housing portion and arranged relative to a proximal end of the illumination light guide; and a laser treatment assembly. The laser treatment assembly includes a laser guide having a laser fiber optic extending through the elongate shaft having a proximal end and a distal end; a laser diode emitter located proximal to the proximal end of the laser fiber optic, the laser diode emitter having a base coupled to an annular casing, the casing having an inner diameter; and a ball lens supported by the annular casing, the ball lens configured to direct a collimated laser beam from the emitter towards the proximal end of the laser guide.

In an interrelated aspect, provided is a method of treating glaucoma of an eye using a handheld intraocular device including penetrating the eye with an elongate shaft of the handheld intraocular device to access a ciliary process; delivering laser energy through the elongate shaft to the ciliary process to effect photocoagulation; and delivering cooled liquid to the eye during delivery of laser energy.

The elongate shaft can be coupled to a disposable portion of the handheld intraocular device, the disposable portion releasably coupled to a reusable portion of the handheld intraocular device. Delivering laser energy can include delivering laser energy from a 810 nm laser diode. The laser diode can be positioned within the reusable portion and transmitted through a junction between the reusable and disposable portions to a laser guide of the disposable portion. The laser diode can be positioned within the disposable portion. Delivering laser energy can include delivering laser energy a distance away from the ciliary process. The distance can be 1-3 mm away from the ciliary process. The method can further include delivering laser energy repeatedly to a plurality of ciliary processes along at least 270 degrees up to 360 degrees of the eye. The method can further include imaging the ciliary process using an imaging assembly transmitting images to an external monitor. The imaging assembly can transmit images wirelessly. The imaging assembly can include an image sensor and a single lens element each contained within the reusable portion. The single lens element can be a Steinheil triplet.

The disposable portion can include an illumination source comprising an LED coupled to a fiberoptic of an illumination light guide. The LED can be a white light LED. The disposable portion can further include one or more additional LEDs. The one or more additional LEDs can emit red, near-infrared, blue, green, ultraviolet, or near ultraviolet light.

The cooled liquid can be delivered through a fluid channel extending within the disposable portion. The fluid channel can extend through the elongate shaft. The cooled liquid can be water or saline. The fluid channel can be configured to deliver a therapeutic agent.

In an interrelated aspect, provided is an intraocular cyclophotocoagulation device having a proximal, reusable portion and a distal, disposable portion. The reusable portion includes a proximal housing portion having a distal end region; a laser treatment assembly comprising a laser diode and configured to direct a collimated laser beam towards the distal end region of the proximal housing portion; and an imaging assembly. The distal, disposable portion includes a distal housing portion having an elongate shaft extending distally from a distal end region of the distal housing portion, the distal housing portion having a proximal end region configured to reversibly couple with the distal end region of the proximal housing portion; a laser guide extending through the elongate shaft and an aspheric lens positioned within the distal housing portion to receive the collimated laser beam from the proximal reusable portion and direct the collimated laser beam toward a proximal end of a fiberoptic of the laser guide; an imaging guide extending through the elongate shaft and an objective lens located at a distal end region of the elongate shaft, the objective lens arranged to focus an image onto a distal end of a fiberoptic of the imaging guide; and an illumination light guide extending through the elongate shaft.

In an interrelated aspect, provided is an endoscopic device for intraocular treatment having a proximal, reusable portion with a proximal housing portion having a distal end region; and a distal, disposable portion with a distal housing portion having an elongate shaft extending distally from a distal end region of the distal housing portion. The distal housing portion has a proximal end region configured to reversibly couple with the distal end region of the proximal housing portion. A frustoconical lens is positioned within a distal end region of the elongate shaft. The device can further include an imaging channel extending within the elongate shaft configured to transmit light from the frustoconical lens. The device can further include one or both of an illumination channel and a laser treatment channel extending within the elongate shaft.

In an interrelated aspect, provided is a method of treating glaucoma using an endoscopic device having an elongate shaft including delivering cold water ab interno through the elongate shaft while applying laser energy to a ciliary body of an eye through the elongate shaft.

In some variations, one or more of the following can optionally be included in any feasible combination in the above methods, apparatus, devices, and systems. More details are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1 is a diagram of the front portion of the eye;

FIG. 2A is a box diagram illustrating an implementation of a handheld cyclophotocoagulation device;

FIG. 2B is a box diagram illustrating the device of FIG. 2A illustrating additional components in the reusable portion;

FIG. 2C is a box diagram illustrating another implementation of the device;

FIG. 3A is a schematic perspective view of a handheld cyclophotocoagulation device;

FIG. 3B is a schematic cross-sectional view of the device of FIG. 3A;

FIG. 3C is a schematic distal end view of the distal tip section of the device of FIG. 3A;

FIG. 4A is a perspective view of an implementation of a handheld cyclophotocoagulation device;

FIG. 4B is a cross-sectional view the device of FIG. 4A taken along line B-B;

FIG. 4C is a perspective distal end view of a device with disposable and reusable portions exploded;

FIG. 4D is a perspective proximal end view of the device of FIG. 4C

FIG. 5A is a side view of the device of FIG. 4A;

FIG. 5B is a partial view of the device of FIG. 5A;

FIG. 5C is a distal end view of the probe of FIG. 5A;

FIG. 6A is a schematic illustrating an objective lens having an hourglass configuration for use with a cyclophotocoagulation device illustrating on-axis light source;

FIG. 6B is a schematic illustrating the objective lens of FIG. 6A illustrating an off-axis light source;

FIG. 7A is a schematic illustrating an objective lens having a capsule-shape configuration for use with a cyclophotocoagulation device illustrating on-axis light source;

FIG. 7B is a schematic illustrating the objective lens of FIG. 7A illustrating an off-axis light source;

FIG. 7C is another schematic view of the objective lens of FIG. 7A;

FIG. 8A is a schematic showing a high numeric aperture optical fiber positioned relative to an LED illumination light source;

FIG. 8B is a schematic showing a low numeric aperture optical fiber positioned relative to an LED illumination light source;

FIG. 9A is an LED illumination light source and optical fiber for positioning within a disposable portion of a device;

FIG. 9B is an implementation of the LED illumination light source and optical fiber of FIG. 9A arranged relative to a spherical cavity filled with optical adhesive creating a focusing lens;

FIG. 9C is an implementation of the LED illumination light source and optical fiber of FIG. 9A arranged relative to a spherical ball lens;

FIG. 10A illustrates an optical chain between disposable and reusable components;

FIG. 10B illustrates the optical chain in the presence of axial misalignment;

FIG. 11 illustrates a laser treatment assembly contained within the disposable portion;

FIG. 12A is a schematic of the short optical path of the imaging assembly showing a single lens element for relaying the image of the fiberoptic onto a subset of the imaging pixels;

FIG. 12B is a schematic illustrating the pixel ratio of the fiberoptic relay for significant oversampling;

FIGS. 13A-13B illustrate an adjustment mechanism for changing the focus of the objective lens.

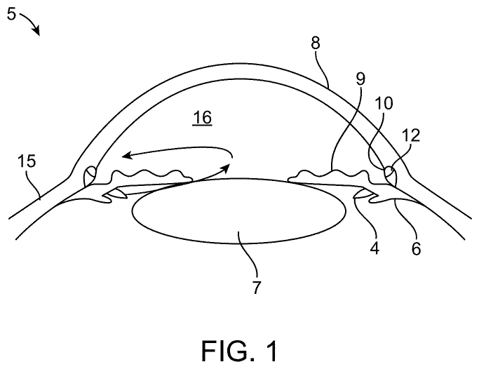

It should be appreciated that the drawings are for example only and are not meant to be to scale. It is to be understood that devices described herein may include features not necessarily depicted in each figure.

### DETAILED DESCRIPTION

Disclosed is a fully hand-held endoscopic photocoagulation (ECP) device for reducing aqueous humor production for the purpose of controlling intraocular pressure (IOP). More particularly and as will be described in detail below, the devices described herein have a reusable portion coupled to a disposable portion. The reusable portion contains components such as the imaging unit, therapeutic laser diode, microprocessor, etc. that are contained within a handle having a small form factor for ergonomic use and that remains outside of the eye. The treatment laser diode transmits in the near-infrared wavelength (810 nm) to photocoagulate (e.g., burn and shrink) the ciliary body to decrease its aqueous humor production within the anterior chamber. The reusable portion reversibly couples to a disposable portion of the handle. The disposable portion includes the distal shaft that is sized for insertion in an eye (e.g., the anterior chamber and/or the posterior chamber between the iris and the capsular bag). The distal shaft contains channels for illumination, imaging, and laser treatment. The disposable portion also contains the illumination source. The design of the illumination source significantly reduces the overall cost and allows for it to be contained within the disposable portion and disposed of after a single use with a single patient. Similarly, the objective lens of the imaging channel in the disposable portion is cheaper and easier to manufacture thereby reducing costs of the disposable portion, but without impacting the optics achieved. Additionally, the disposable portion contains optics that when connected to the reusable portion maximizes efficiency of transmission of the collimated laser light from the treatment laser diode into the treatment channel even in the presence of misalignment between the disposable and reusable portions upon attachment.

Use of the terms “hand piece” “hand-held” or “handle” herein need not be limited to a surgeon's hand and can include a hand piece coupled to a robotic arm or robotic system or other computer-assisted surgical system in which the user uses a computer console to manipulate the controls of the instrument. The computer can translate the user's movements and actuation of the controls to be then carried out on the patient by the robotic arm.

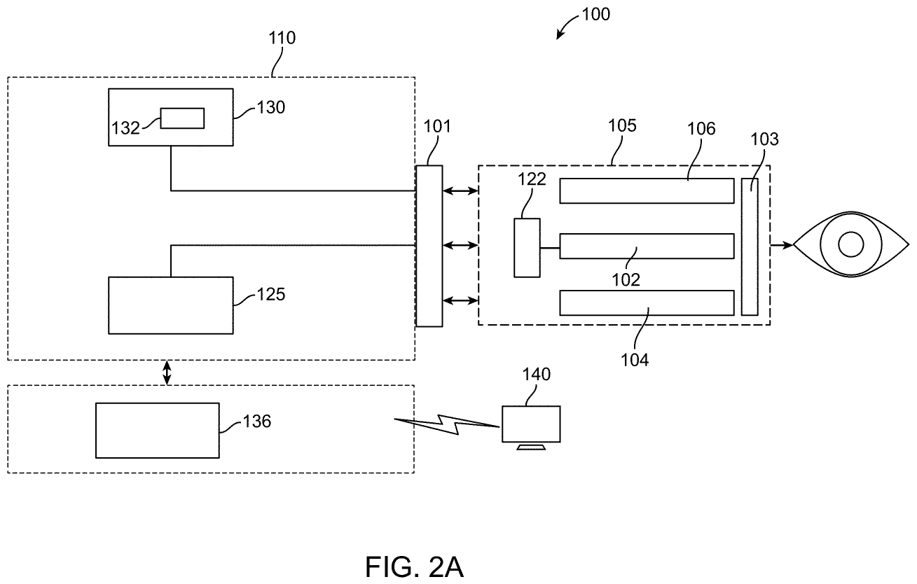

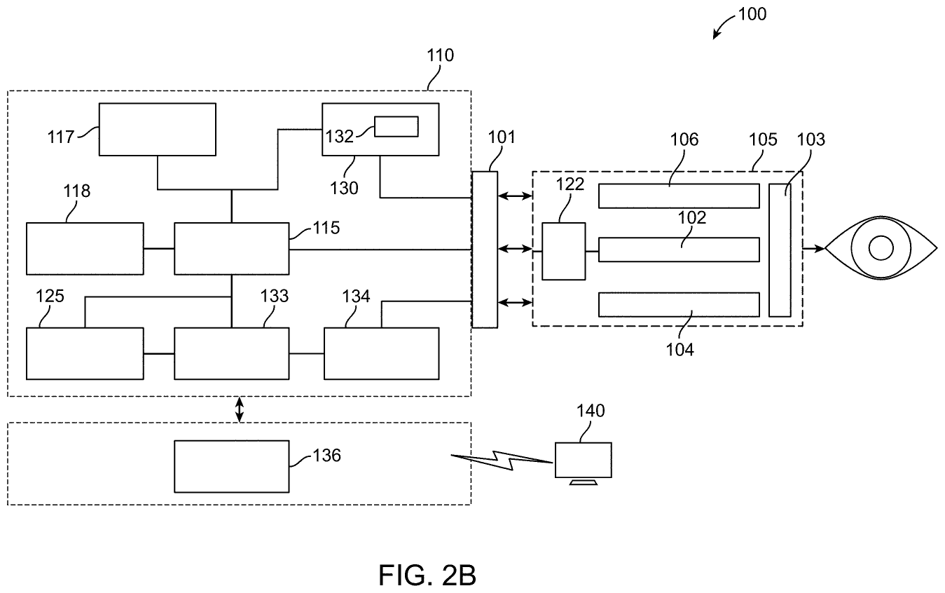

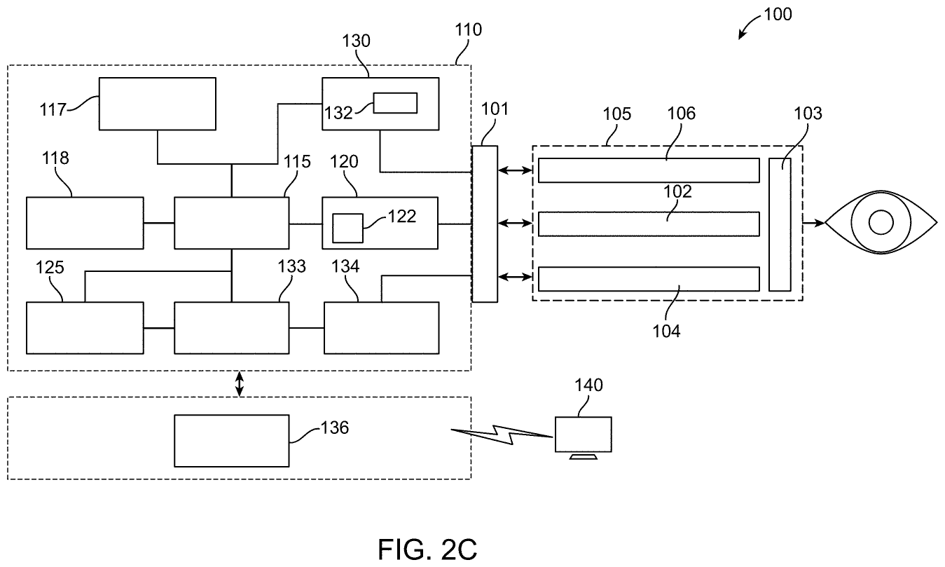

FIG. 2A is a box diagram illustrating an implementation of a handheld cyclophotocoagulation device 100 including a distal disposable portion 105 configured to releasably couple to a proximal reusable portion 110 via a coupling mechanism 101. The disposable portion 105 generally includes the components of the device configured to come into contact with the patient including a distal elongate probe shaft 103 configured to insert within the eye. The distal elongate probe shaft 103 of the disposable portion 105 encloses the guides, which can be fiberoptic bundles or single fiber optics, including a light guide/lens 102, an image guide/lens 104, and a laser guide/lens 106. The reusable portion 110 generally includes the components of the device 100 that are configured to remain outside the patient such as the laser generator and video camera. The reusable portion may be re-sterilized and reused, whereas the disposable portion 105 is manufactured of lower cost materials such that it is financially practical for the disposable portion 105 to be thrown away after a single use.

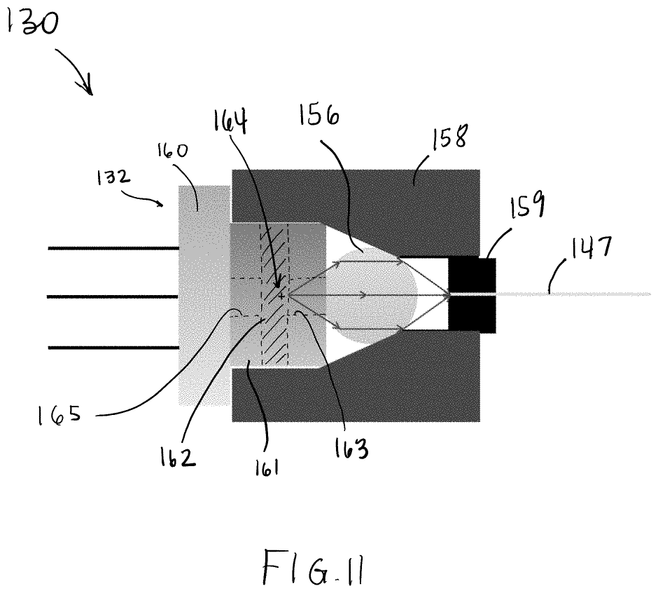

The reusable portion 110 includes one or more components that control the operation of the device 100 including one or more of the illumination assembly 120, the imaging assembly 125, the laser treatment assembly 130 and/or any focusing mechanisms 133 and collimating lenses 134 for the laser treatment assembly 130 and/or imaging assembly 125. The arrangement of the component parts between the two portions 105, 110 can vary. For example, FIG. 2B is a box diagram of an implementation of the device 100 having an illumination light source 122 of the illumination assembly 120 in the disposable portion 105. FIG. 2C is a box diagram of another implementation of the device 100 having the illumination light source 122 of the illumination assembly 120 in the reusable portion 110. The illumination light source 122 as well as the laser treatment assembly 130, including both the laser diode 132 and the focusing lens, can be positioned within the disposable portion 105 so as to avoid having the laser light and illuminating light crossing the junction between the two housing component. This will be described in more detail below with regard to FIG. 11. Regardless the arrangement, the coupling mechanism 101 between the disposable portion 105 and the reusable portion 110 minimizes losses in visible light and laser light transmission to and from the distal end of the disposable portion 105 and the reusable portion 110. The system is described herein having three separate channels for laser, imaging, and illumination. The system can also combine two of the three channels into a single channel for a total of two channels. For example, the laser guide/lens 106 and the imaging guide/lens 104 can be combined into a single channel and the light guide/lens 102 can be a separate channel of the system.

Each of the components will be described in more detail below.

Although described in the context of cyclophotocoagulation using 810 nm wavelength laser energy, the device 100 can be used for a variety of medical laser treatments. The device can also incorporate one or more light sources, in the disposable and/or reusable portions, of any of a variety of wavelengths for performing other treatments. For example, one or more LEDs or laser diodes and corresponding fiber optics can be incorporated within the disposable portion to perform photobiomodulation including red (600-700 nm), near-infrared (770-1200 nm), blue, green, ultraviolet or near ultraviolet, or other colors.

Reusable Portion

As mentioned above, the reusable portion 110 includes the components such as the imaging assembly 125 and the laser treatment assembly 130 and couples to the disposable portion 105 that includes the distal shaft 103 enclosing the light guide 102, image guide 104, and laser guide 106 for positioning directly within an eye of the patient. The coupling between the two enables, for example, laser energy transmission from the laser diode 132 of the laser treatment assembly 130 in the reusable portion 110 into the laser guide 106 of the disposable portion 105 into the eye. Similarly, image data from within the eye is transmitted through the image guide 104 of the disposable portion 105 to the imaging assembly 125 in real-time during laser treatment. The disposable portion 105 can also include an illumination light source 122 that upon coupling with the durable reusable portion 110 is powered by the illumination assembly 120 to deliver light through the light guide 102 to illuminate regions within the eye that are being treated. The result is a fully hand-held endoscopic photocoagulation device that is a small form factor and able to be manufactured in a more cost-effective manner without negatively impacting the optics and treatment effectiveness.

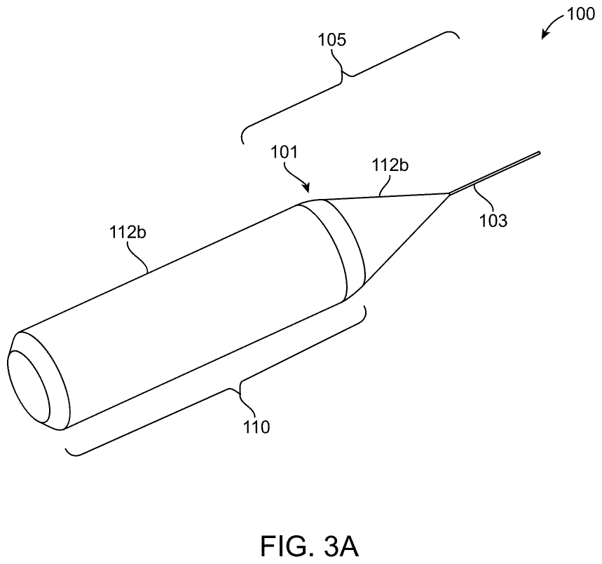

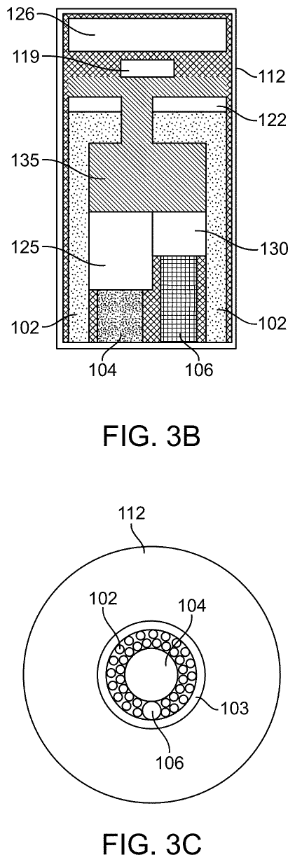

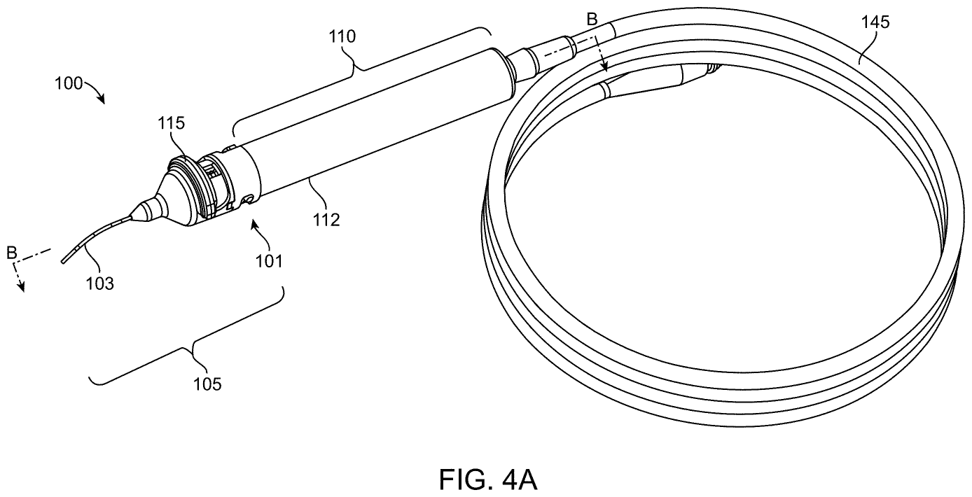

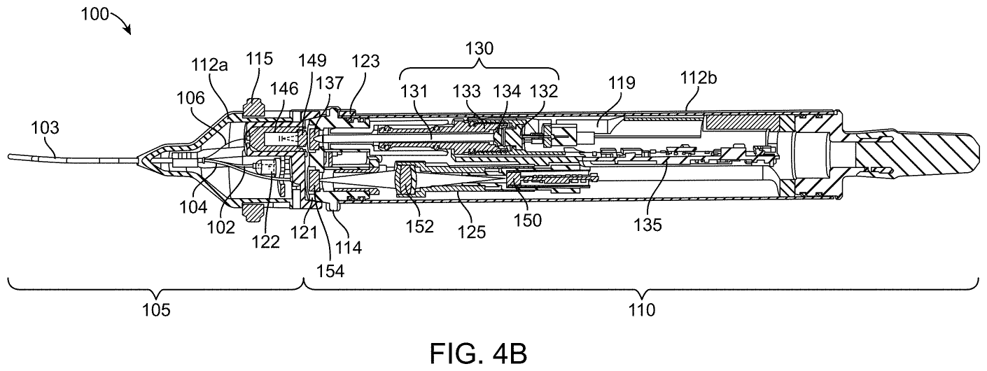

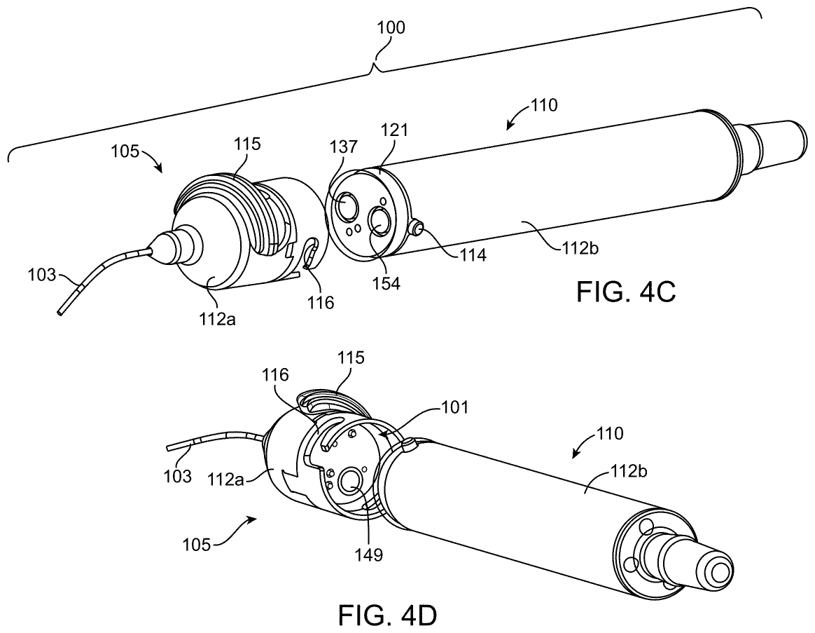

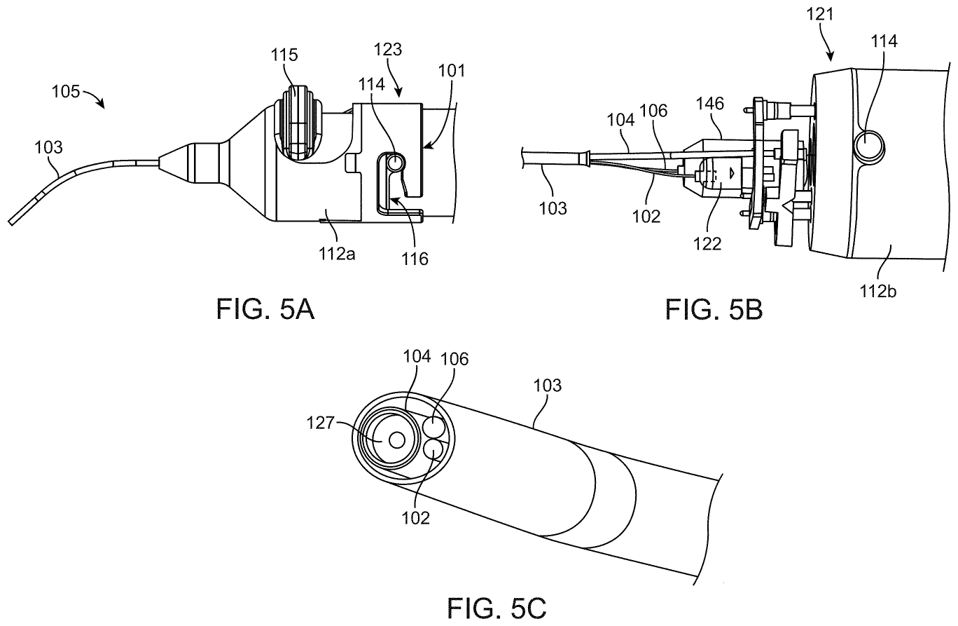

FIG. 3A is a schematic perspective view of the device 100 including disposable and reusable portions 105, 110. FIG. 3B is a schematic cross-sectional view of the device of FIG. 3A illustrating components within the device 100. FIG. 3C is a schematic distal end view of the distal tip section of the device of FIG. 3A. FIGS. 4A-4B, 4C-4D, 5A-5C are views of another implementation of a handheld device 100.

As best shown in FIG. 4B, the imaging assembly 125 includes an image sensor 150 and a single lens element 152 separated from the image sensor and supported by a frame or bracket. The image sensor can be an imaging CCD or CMOS sensor. The imaging assembly 125 can additionally incorporate a glass window 154 (e.g., sapphire window) or other optically clear element at a distal end region of the housing portion configured to seal the housing portion and prevent introduction of contaminants from the environment. The imaging assembly 125 is positioned within the reusable portion 110 so as to receive an endoscope image from the fiberoptic bundle of the image guide 104 within the disposable portion 105. Upon coupling, the imaging assembly 125 in the reusable portion 110 can focus on the proximal end of the image guide 104 within the disposable portion 105.

In conventional endoscopes, an image is transmitted through an image fiber bundle with a fixed number of individual fibers nf. The image is present on the proximal end of the image fiber bundle with an image diameter d. The image diameter d is conventionally the same as the diameter of the fiber bundle as the intent of conventional endoscope optics is to project the image using the maximum sensor area. Standard solutions in microscope optics is to magnify the image on the small fiber bundle end to fill the frame of the image sensor or to project an enlarged virtual image into a viewing eyepiece. This requires multiple optical elements that are relatively large and expensive. The image brightness is significantly reduced due to the magnification to fill the large relative area of the camera sensor due to inverse square law.

In the present device relay uses 1:1 relay lens element 152 and a small subsection of the total pixels available in the image sensor 150. Each individual fiber is relayed 1:1 onto a subset of total pixels of the image sensor 150. The pixel oversampling ratio can be at least 9:1 for image enhancement and oversampling in which 9 pixels of the image sensor pixel array are covered by a single fiber of the fiber bundle. The pixel oversampling ratio can be between 4:1 and 25:1 including 4:1, 9:1, 16:1, and 25:1. The subset usage relies on the pixel-to-bundle ratio as well as the size of pixels on the fiber bundle and the size of pixels on the image sensor 150. The sensor pixel size can be 1-3 μm², preferably about 1.2-1.5 μm².

The image guide 104 includes an objective lens 127, which will be described in more detail below that focuses an image on the distal end of the fiberoptic bundle. The imaging fiberoptic bundle contains thousands of individual fiberoptics that are each analogous to a pixel of the image. The image is transmitted through the fiberoptic bundle to the proximal end of the fiberoptic bundle. In conventional systems, the proximal end of the fiberoptic bundle is arranged to transmit the image through a series of three lenses. The image sensor of the imaging assembly, which is positioned an optical path length away from the proximal end of the fiberoptic bundle, receives the magnified projected image. The projected image diameter on the sensor in these conventional systems may be magnified 2× the fiberoptic image diameter. In the present device, the proximal end of the fiberoptic bundle is arranged to transmit the image through the single lens element 152 of the imaging assembly 125 having an optical path length that is about ½ as long as the optical path length of the three-lens system described above. The image sensor 150 of the imaging assembly 125, which is positioned away from the proximal end of the fiberoptic bundle by the shorter optical path length, receives the projected image. The projected image is not magnified and is instead a 1:1 relay such that the projected image diameter on the sensor is the same as the fiberoptic image diameter. Because the projected image on the fiber in the present devices is not magnified, according to the inverse square law, the projected image is 4× as bright as conventional systems using a three-lens system and having an optical path length that is twice as long. Thus, there is no loss of brightness in the imaging assembly 125 and is about 4× brighter compared to conventional microscopic endoscopes. The imaging fiber numeric aperture can be about 0.2-0.9, preferably about 0.3-0.5. The imaging fiber diameter can be about 300-2000 μm, preferably about 600-700 μm. The optical path length from the proximal end of the fiber to the location of the image sensor 152 can be about 20-100 mm, preferably about 30-40 mm. The lens diameter can be about 3-30 mm, preferably about 5-10 mm.

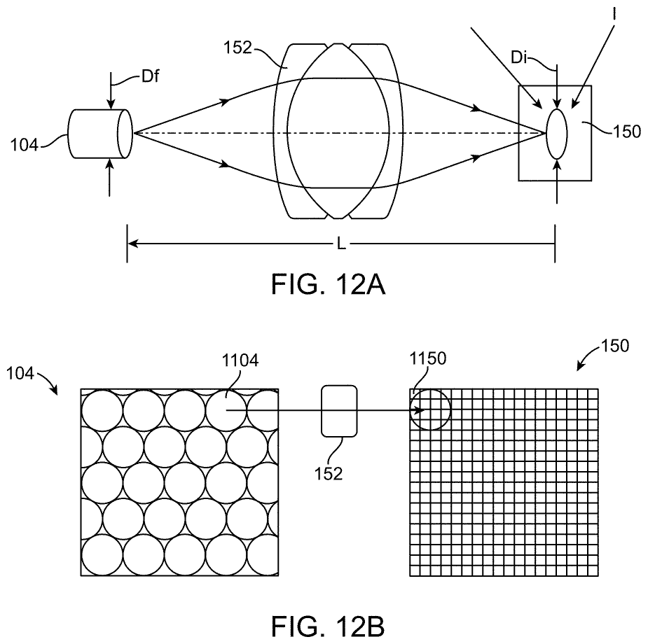

The single lens element 152 of the imaging assembly 125 can be a compound triplet lens formed of three lenses cemented together forming a single lens element 152 configured to relay at a desired ratio. The triplet lens element 152 can correct for chromatic aberration at three wavelengths. In some implementations, the lens element 152 is a Steinheil triplet that is designed to relay at a 1:1 ratio. FIG. 12A is a schematic of the short optical path of the imaging assembly showing a single lens element for relaying the image of the fiberoptic onto a subset of the imaging pixels. A single lens element 152 is positioned between the proximal end of the fiberoptic bundle of the image guide 104 and the image sensor 150. The lens 152 illustrated is a Steinheil triplet having a relatively short optical path length L. The image I projected onto the image sensor 150 has a diameter Di that is the same diameter as the fiber image diameter Df (i.e., 1:1 relay). Each individual fiber of the fiberoptic bundle of the image guide 104 is relayed 1:1 by the Steinheil lens onto, for example, 16 sensor pixels of the image sensor 150 as discussed above. FIG. 12B is a schematic illustrating the pixel ratio of the fiberoptic relay for significant oversampling. The schematic includes a close-up section of the fiberoptic bundle of the image guide 104 made up of individual fibers 1104. The image from the fiber 1104 is projected through a lens element 152 onto an image sensor 150, which is an array of pixels 1150. Each pixel 1150 of the sensor pixel array can be about ¼ as wide as 1 individual fiber 1204. Each individual fiber 1104 is relayed 1:1 by the single lens element 152 onto a sub-set of sensor pixels 1150. In the example of FIG. 12B, one fiber 1104 projects onto 16 sensor pixels 1150. This allows for significant oversampling for image fractal smoothing and quality enhancement, particularly for anti-aliasing. There is no loss of data due to low contrast or undersampling. The Steinheil triplet is also desireable in that it can correct for all 5 Seidel aberrations including spherical aberration, coma, astigmatism, distortion, and Petzval field curvature. The triplet lens element 152 can be designed to achieve a particular relay ratio that need not be 1:1. For example, the pixel size can be even smaller (e.g., 0.5 micron pixel size) such that the image projected by the lens element 152 is smaller than real life or 1:0.5 pixel ratio. The single lens element 152 can be designed to have any desired relay ratio, particularly where sensor size is of no concern, just sensor density to achieve better brightness and better contrast with less power requirement.

Again with respect to FIGS. 4A-4B and also 4C-4D, the laser treatment assembly 130 can include a laser diode 132 with collimating optics. In some implementations, the laser treatment assembly 130 is positioned within the reusable portion 110 (see FIG. 4B). The laser diode 132 is positioned near a focusing mechanism 133 incorporating a collimating lens 134 and light tube 131 (see FIG. 4B). The collimating lens 134, which can be a high numeric aperture collimating lens, is positioned a collimating distance away from the laser diode 132 and allows for a narrower beam width of the laser diode 132. The high numeric aperture collimating lens 134 allows for close placement to the laser diode 132. The focusing mechanism 133 can be a threaded element that adjusts the position of the collimating lens 134 and the light tube 131 relative to the laser diode 132 at the time of manufacturing and assembly to ensure proper arrangement of the components. Within the collimating stage of the optical chain of the laser treatment assembly 130, the diameter of the resultant beam is less than the mating window and clear aperture of the collimating lens 134. Preferably, the beam width is about ½ the diameter of the clear aperture of the collimating lens 134 or less. Collimation is tolerant of lateral variance in emitting point in laser diode 132 and is set to optimize beam width based on Z-axis position of the laser diode 132. The laser beam emitted from the laser diode 132 travels through the collimating lens 134 and the light tube 131 towards an optical window 137 positioned near a distal end region of the reusable portion 110. The collimated beam crosses the junction largely unscathed with minimal divergence or convergence across the junction and the window 137. The optical window 137 can be a sapphire window positioned within a distal end region of the housing 112 of the reusable portion 110 to seal the housing portion from contaminants. The material of the sapphire window 137 upon good window alignment has no effect on the beam path. The beam is directed out the distal end of the reusable portion 110 into the proximal end of the disposable portion 105. The disposable portion 105 incorporates focusing optics, which will be described in detail below that ensures even if the beam is misaligned, the marginal rays of the collimated laser beam are directed into the fiberoptic of the laser guide 106.

The collimating lens 134 can have a numeric aperture of about 0.3-0.8, preferably about 0.5-0.7. The collimating lens 134 can have a diameter of about 2-10 mm, preferably about 3-6 mm. The collimating lens 134 can have a focal length that is about 1-4 mm, preferably about 2-3 mm.

The laser treatment assembly 130 can also be positioned within the disposable portion 105, which will be described in more detail below with regard to FIG. 11.

The reusable portion 110 can incorporate one or more cooling features 118 configured to cool the internal components positioned within the housing 112. The cooling feature 118 can include a heat sink 119 (see FIG. 3B or 4B) to provide conductive cooling, an active cooling fan 126 (see FIG. 3B), heat pipe, or fluid heat exchanger depending on the overall power of the laser incorporated in the device 100.

The reusable portion 110 of the device can include a microprocessor/controller 136 on a PCB board 135 powered by a power system. The PCB board 135 translates user inputs to actively control one or more of the imaging assembly 125, the laser treatment assembly 130, any active cooling components such as an active cooling fan 126, if present.

The PCB board 135 can include a communication assembly configured to be in operative communicate with one or more peripheral devices such as an external computing device 140 having a video monitor. The connection can include a wired communication port such as a RS22 connection, USB, Fire wire connections, proprietary connections, or any other suitable type of hard-wired connection configured to receive and/or send information to an external computing device or ablation device. The communication assembly can also include a wireless communication port such that information can be fed between the device and the external computing device 140 and/or device via a wireless link, for example, to display information in real-time on the external computing device 140 about operation and/or control programming of the device 100. It should be appreciated that the external computing device 140, such as a console or handheld device such as a tablet, can communicate directly to the device 100. Any of a variety of adjustments to and programming of the device can be performed using the external computing device 140. The wireless connection can use any suitable wireless system, such as Bluetooth, Wi-Fi, radio frequency, ZigBee communication protocols, infrared, or cellular phone systems, and can also employ coding or authentication to verify the origin of the information received. The wireless connection can also be any of a variety of proprietary wireless connection protocols.

The external computing device 140 can be a video monitor and/or computing device 140 for use in real-time during a procedure. The imaging guide 104 can relay images to its proximal end within the disposable portion 105 to the imaging assembly 125 within the reusable portion 110, which is interfaced with a video camera via cable 145 and the video data transmitted to the monitor. The reusable portion 110 can include a video transmitter, which can be a wireless transmitter. The video transmission can be accompanied by control, status, and/or user information. The external computing device 140 can be equipped with recording and image processing software such that the images can be computer-enhanced in real-time before they are displayed on the monitor (e.g., image size, contrast, color balance, orientation). In some implementations, the PCB can incorporate an accelerometer so that the image data is automatically adjusted for direction in real-time so that the video projected to a user is shows which way is “up”.

Power can be supplied to the reusable portion 110 of the device 100 such as via the cable 145 extending from a proximal end of the reusable portion 110. The cable 145 may also be configured to connect the device 100 to a wall socket. The device 100 can also be powered by one or more batteries. The battery can be incorporated within a region of the durable portion 110, either internally or coupled to a region of the housing such as within a modular, removable battery pack. The battery can have different chemical compositions or characteristics. For instance, batteries can include lead-acid, nickel cadmium, nickel metal hydride, silver-oxide, mercury oxide, lithium ion, lithium ion polymer, or other lithium chemistries. The device can also include rechargeable batteries using either a DC power-port, induction, solar cells, or the like for recharging. Power systems known in the art for powering medical devices for use in the operating room are also to be considered herein such as spring power or any other suitable internal or external power source.

The device 100 can incorporate automated power modulation feature by implementing clinical decision support algorithms. Machine learning and machine vision frameworks for ophthalmic treatments using lasers can be incorporated. The machine learning framework can use any appropriate Segmentation, Feature extraction, image recognition or image processing algorithm. Some examples include deep learning models such as Convolutional Neural networks, U-NET systems for image segmentation, MASK-R Convolutional Neural Networks for image segmentation, RANSAC Algorithms for change and outlier detection; Bag-Of-Features algorithms to assign and detect feature vectors in images, Autoencoders for feature detection, and a great many other Regression and Classifier algorithms. The framework can implement an image recognition and processing algorithm allowing for the analysis of image data acquired during a procedure. Laser energy applied by the devices described herein is designed to damage tissues, but too much laser energy applied during a procedure in the eye can cause unwanted damage. The imaging assembly can communicate information to a control algorithm that adjusts the power to the laser treatment assembly automatically to prevent the sort of unwanted tissue damage while allowing the desired tissue damage for treatment of the disease.

A machine learning image detection algorithm can identify the ciliary body or ciliary bodies in an image communicated from the imaging assembly. The algorithm can be a trained Deep Neural Network or Convolutional Network or similar. The algorithm can segment the image and detect a pattern associated with a ciliary process with high probability. A second algorithm (e.g., a “feature extraction algorithm”) can characterize the shape and size of each ciliary process in the image. The Feature Extraction Algorithm can use any number of machine vision and machine learning techniques to orient and characterize the shape (e.g., proportions, aspect ratio, size and shape relative to neighboring ciliary process, counting pixels, etc.). As the laser power is applied to the ciliary process in the field of view, the machine vision can continuously monitor the images for changes in the shape, size, aspect ratio or other relevant feature and automatically and very quickly reduce the power at the appropriate moment (e.g., after sufficient shrinkage and prior to ciliary process excessive damage) thereby preventing the undesireable laser treatment effect. The algorithm(s) assists the doctor in titrating the laser power applied to the ciliary tissue to an appropriate level to achieve the desired treatment result.

Disposable Portion

Again with respect to FIGS. 3A-3C, 4A-4B, and 4C-4D, the disposable portion 105 includes a distal shaft 103 designed to insert within the anterior chamber, the posterior chamber, and/or the vitreous chamber of the eye. The shaft 103 is preferably 18-, 19- or 20-gauge for use within the anterior and posterior chambers although other sizes are considered. For example, larger gauges (23-, 25-) can be used for retinal indications or when used within the vitreous chamber. The shaft 103 can be straight as shown in FIG. 3A or curved as shown in FIG. 4A. The curvature of the shaft 103 can allow for a greater field of view (FOV) depending on the indication. The FOV can be between 45 and 150 degrees, preferably about 130-140 degrees.

The shaft 103 encloses channels running therethrough including the illumination channel or light guide 102, imaging channel or image guide 104, and laser treatment channel or laser guide 106 for positioning directly within an eye of a patient. The channels can include one or more fiberoptics or fiberoptic bundles that extend from a distal end of the shaft 103 to a proximal end region of the disposable portion 105. Each fiberoptic or fiberoptic bundle can include a central core surrounded by cladding material. The image guide 104 can include an objective lens 127 at a distal end of the shaft 103 and fiberoptic bundles that preserve the information in the optical images being transmitted (e.g., coherent bundles). The proximal end of the image guide 104 interfaces with the imaging assembly 125 of the reusable portion 110. The light guide 102 can include fiberoptic bundles that can be incoherent in that they don't retain the relative position. The laser treatment guide 106 can be a single, small diameter optical fiber that is designed to deliver the laser photocoagulation radiation (typically 810 nm infrared radiation) from the laser source in the reusable portion 110 to the distal treatment end of the shaft 103. Shorter wavelength laser sources can also be used (e.g., green 532 nm, yellow 577 nm).

The channels of the shaft 103 in the disposable portion 105 are configured to reversibly interface with the corresponding assembly in the reusable portion 110. Each of the disposable portion 105 and the durable portion 110 can include a housing portion 112a, 112b that are arranged to couple together using a coupling mechanism 101. The housing portions 112a, 112b can be formed of a relatively rigid, lightweight material(s) preferably made of a plastic, but can also be metal. The coupling mechanism 101 can vary including bayonet, threads, snap-lock, and the like.

FIGS. 5A-5B shows an implementation of a coupling mechanism 101 incorporating a release button 114 on a distal end 121 of the reusable portion 110 housing 112b configured to engage a corresponding slot 116 on a proximal end 123 of the disposable portion 105 housing 112a. The distal end 121 of the durable portion housing 112b is sized and shaped to be received within the proximal end 123 of the disposable portion housing 112a. The proximal end 123 of the disposable portion housing 112a can surround the distal end 121 of the durable portion housing 112b so that the taper of the distal end 121 wedges with a corresponding shaped region within the proximal end 123 of the disposable portion housing 112a. The release button 114 on the distal end 121 can insert within and engage the corresponding slot 116 in the proximal end 123 of the disposable portion housing 112a to ensure the coupling. The taper of the distal end 121 of the reusable portion housing 112b can be spring-loaded providing a snug, wedged mating between the two portions 105, 110 and aids in achieving optimum attachment and alignment of the components within the two portions 105, 110.

The coupling mechanism 101 between the two housing portions 112a, 112b can be mechanical as well as electronic. For example, the electronic coupling ensures if the disposable portion 105 includes the illumination light source 122 (e.g., white light LED) it interfaces appropriately with electronics in the durable portion 110 (e.g., LED driver) that power the illumination light source 122 positioned in the reusable portion 110. The coupling mechanism 101 ensures the imaging assembly 125 of the reusable portion 110 is axially aligned and optically connected with the image guide 104 of the disposable portion 105 and the laser treatment assembly 130 of the reusable portion 110 is axially aligned and optically connected with laser guide 106 of the disposable portion 105, which will be discussed in more detail below.

As mentioned above, the image guide 104 can include a fiberoptic bundle having a proximal end and a distal end, the distal end positioned near an objective lens 127 at a distal end of the shaft 103 (see FIG. 5C). The objective lens 127 is designed to capture a high-resolution tissue image by focusing the image onto the distal end of the imaging fiberoptic bundle, which is then transmitted to the proximal end. The field of view (FOV) of the objective lens 127, because it is designed to be positioned inside the eye, is greatly increased compared to surgical microscopes having limited FOV. The objective lens 127 can have a depth of focus that is manually or automatically adjustable between 0.75-40 mm, or 1-30 mm depth of focus, or preferably 1-6 mm.

The majority of microendosopic objective lenses (e.g., used for 1-6 mm working distance) incorporate an opaque disc having a central aperture to increase the depth of focus (DOF). Increased DOF is a result of primarily paraxial rays being focused via the pinhole effect. Non-paraxial and marginal rays are blocked by the opaque material of the disc. The disc is difficult to manufacture in that the aperture must be placed in the optical path using cumbersome and expensive processes. Typically, a doublet is formed using two glass elements bonded together with the opaque disc sandwiched between them. The aperture in the disc is typically in the sub-millimeter diameter range as small as about 0.15 mm in diameter relative to the working distance. The overall lens diameter is frequently less than about 0.5 mm, which is at the limits of manufacturability.

The endoscopic devices described herein for intraocular treatment can have a proximal, reusable portion with a proximal housing portion having a distal end region and a distal, disposable portion with a distal housing portion having an elongate shaft extending distally from a distal end region of the distal housing portion. The distal housing portion has a proximal end region configured to reversibly couple with the distal end region of the proximal housing portion. A frustoconical lens can be positioned within a distal end region of the elongate shaft acting as an objective lens for imaging. The device can further include an imaging channel extending within the elongate shaft configured to transmit light from the frustoconical lens. The device can further include one or both of an illumination channel and a laser treatment channel extending within the elongate shaft.

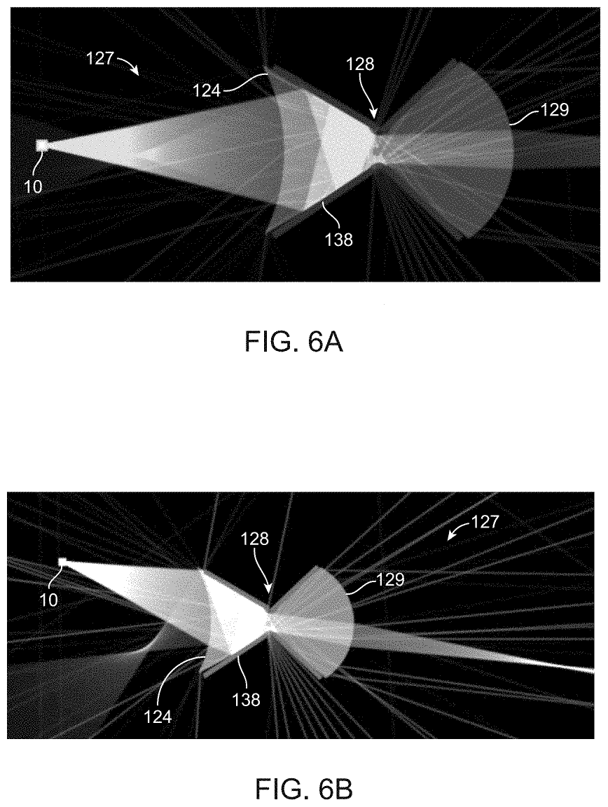

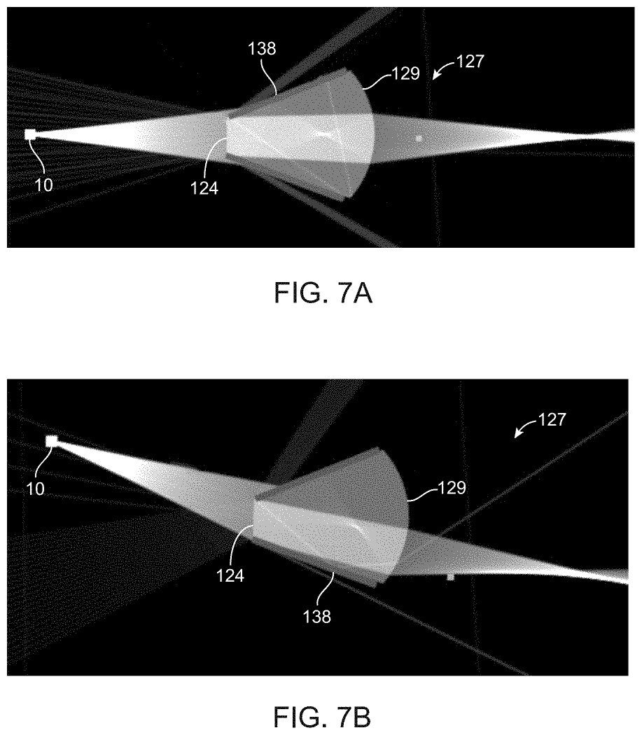

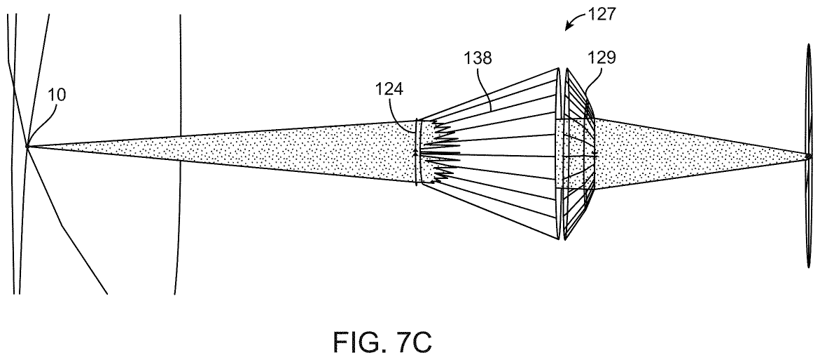

The image guide 104 of the devices described herein can incorporate an objective lens 127 that has an apertureless configuration and is manufactured as a single piece. FIGS. 6A-6B and FIGS. 7A-7C are schematics illustrating objective lenses 127 manufactured as a monolithic component and without an aperture. The objective lens 127 of FIGS. 6A-6B has an hourglass configuration and the objective lens 127 of FIGS. 7A-7B has a frustoconical configuration. The hourglass configuration of the objective lens 127 places a neck 128 with a small diameter in the optical path that allows only paraxial rays to propagate. The neck 128 is similar in size to what the aperture in the opaque disc of conventional objective lenses (e.g. about 0.10 mm-0.15 mm). The entry surface 124 positioned distal to the neck 128 can be aspheric, spherical, or another optimized lens shape. The exit surface 129 positioned proximal to the neck 128 can be convex or planar. The walls of the entry surface 124 of the lens 127 approaching the neck 128 have a shape such that the total internal reflection (TIR) is induced in rays from a light source 10 not axially aligned with the neck 128 (see FIG. 6B). The TIR reflects those rays back out of the entry surface 124 preventing non-paraxial and marginal rays from passing through the neck 128. The diameter of the neck 128 determines the extent of non-paraxial ray transmission. The monolithic design is easier to manufacture by machining, diamond turning, injection molding, or similar techniques in that there is no need for the opaque disk aperture or sandwiching of the disc between glass components. The objective lens 127 relies on TIR to reject the non-paraxial and marginal rays from the optical path.

Now with respect to FIGS. 7A-7C, the frustoconical shape configuration of the objective lens 127 has a smaller diameter entry surface 124 and an increasing diameter body moving proximally to a maximal diameter exit surface 129. The entry surface 124 can be planar, convex or concave, aspheric, spherical or other optimized shape lens. The entry surface 124 is positioned distally relative to the exit surface 129 such that the exit surface 129 of the objective lens 127 is located near the distal end of the imaging fiber optic and the entry surface 124 is located away from the fiber optic towards a target. The exterior surface 138 of the lens 127 can be opaque or rough or both. The opaque exterior surface 138 can be due to an exterior coating, exterior diffusive texture or a second shot of opaque polymer creating an exterior profile for the lens 127, for example, a cylindrical profile or other shapes profile appropriate to fit within the system. The exit surface 129 can form the maximum diameter of the lens 127 and can be planar, convex or concave, aspheric, spherical or other optimized shape lens. The small diameter of the entry surface 124 relative to the working distance of the lens 127 allows only paraxial rays to propagate to the exit surface 129 resulting in greatly enhanced depth of field performance. The monolithic design is easier and cheaper to manufacture and does not require an aperture be placed in the optical path.

The method of creating surface opacity of the lens 127 having an hourglass shape can include EDM surface texturing whereas the method of creating surface opacity of the lens 127 having the frustoconical shape can be by applying black high solids ink. Machine surface roughening, EDM surface texturing, black coating using paint, ink, high solids epoxy, etc. or by second shot injections with opaque material are all considered herein.

The clear aperture of the entry surface 124 of the objective lens 127 having the hourglass shape can be about 300-1000 preferably about 500-700 The diameter of the neck 128 can be about 120-500 preferably about 100-300 The clear aperture of the exit surface 129 of the objective lens 127 can be about 300-2000 preferably about 500-700 The index of refraction of the material can be about 1.45-1.7, preferably about 1.65-1.68. The half angle of conical reduction of the objective lens 127 can be about 20°-40°, preferably about 20°-25°. The parameters of the objective lens having the frustoconical shape can be similar. The clear aperture of the entry surface 124 of the objective lens 127 having the frustoconical shape can be about 120-1000 preferably about 100-300 The clear aperture of the exit surface 129 of the objective lens 127 can be about 300-5000 preferably about 500-700 The index of refraction of the material can be about 1.4-1.7, preferably about 1.65-1.68. The half angle of conical expansion of the objective lens 127 can be about 20°-80°, preferably about 40°-50°.

The shaft 103 can also include a light guide 102 arranged relative to an illumination source 122. Conventional endoophthalmic scopes include an illumination source within a remote console. In the devices described herein, the light source 122 can be within the reusable portion 110 and preferably within the disposable portion 105 to provide wide-field illumination within the eye. The light source 122 upon coupling with the durable reusable portion 110 is powered by the illumination assembly 120 and delivers illuminating light through the light guide 102 to illuminate regions within the eye that are being treated. The light source 122 can be an LED and the light guide 102 is an optical fiber 141. It is difficult and expensive to efficiently couple the luminous output of an LED with an optical fiber. The light emitted by the LED die diverges widely, for example with a 120 degree or greater viewing angle (i.e., the light that is at an off-axis angle with 50% or less light intensity) and typically a Gaussian intensity profile. This widely diverging output makes it difficult to collimate and/or focus the light, which in turn, is difficult to inject the light output into an optical fiber for endoscopic illumination. Traditionally, multiple lenses including exotic and expensive cone or parabolic lenses are used to achieve the light injection with relatively low efficiency.

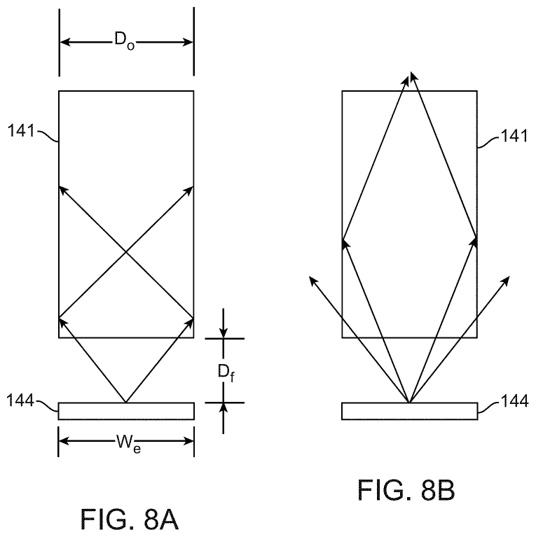

The light source 122 and the optical fiber 141 of the devices disclosed herein are coupled in an efficient and cost-effective manner so that they may be incorporated within the disposable portion 105 of the device 100. Various parameters are considered in determining the optimum potting configuration of the optical fiber 141 and the light source 122 including the numeric aperture NA of the optical fiber 141, which is equivalent to the allowable input angle, the minimum practical distance Df between the optical fiber 141 and the emitter die 144 of the light source 122, the diameter Do of the optical fiber core, the size of the emitting die 144 of the light source 122, and the refractive index of the optical adhesive and/or potting compound used to affix the fiber 141 on or near the emitting die 144 (see FIGS. 8A-8B). In some implementations, the disposable portion incorporates multiple color LEDs (e.g., white, blue, green, far-IR, near-IR, UV, etc.). The multiple light sources of different frequencies can each be affixed on or near a fiber. The various LEDs can be incorporated within the shaft without changing the overall OD of the shaft. FIG. 8A is a schematic showing a high numeric aperture optical fiber 141 positioned relative to an LED illumination light source 122. FIG. 8B is a schematic showing a low numeric aperture optical fiber 141 positioned relative to an LED illumination light source 122. The larger numeric aperture fibers have larger acceptance angles and collect more of the light generated by the light source 122. The optical fiber 141 is ideally positioned directly on top of the die 144 so the distance Df between them is 0. However, other component parts of the light source 122 (e.g., electrical leads, reflector cups, support structures, and the like) prevent this sort of positioning. The minimum distance Df is defined as a repeatable distance that is achievable without damaging the light source 122. The fiber core size relative to the dimensions of the LED emitter die 144 determines the theoretic maximum efficiency. The smaller emitter die 144 allows for greater injection efficiency for a given fiber numeric aperture, diameter, and distance from the die 144.

The numeric aperture of the optical fiber 141 for the light guide 102 can be about 0.1-0.6, or about 0.15-0.5, preferably about 0.5. The dimensions of the LED emitter die 144 can be about 200-1000 μm, about 200-500 μm, preferably about 100-300 μm. The distance Df between the fiber 141 and the die 144 can be about 0-500 μm, about 50-400 μm, preferably about 100-200 μm. The diameter Do of the optical fiber core can be about 10-1000 μm, 25-500 μm, preferably about 100-300 μm. The refractive index of the potting compound can be about 1.4-1.7, preferably about 1.60-1.65.

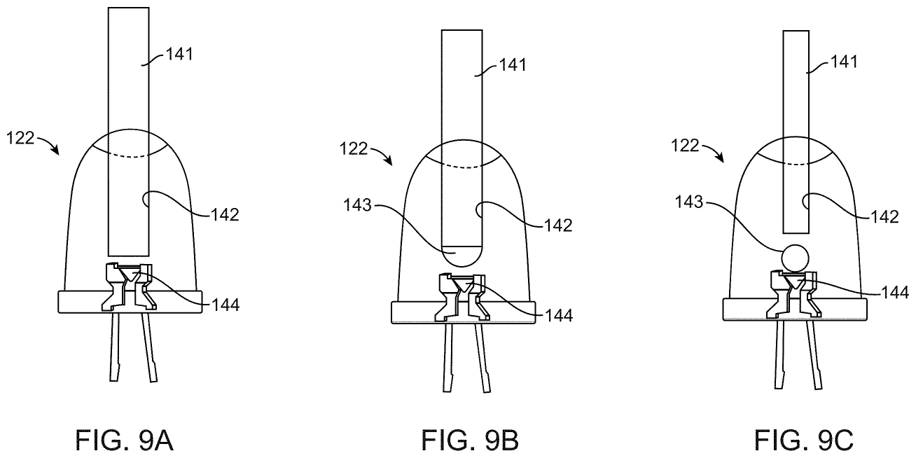

The optical fiber 141 can be potted in a shaft or recess 142 of the light source 122, for example, one created on a surface mount or through-hole LED (see FIG. 9A). The recess 142 can be spherical and filled with an optical adhesive creating a focusing lens (see FIG. 9B). The recess 142 can incorporate a spherical optical lens 143 used for focusing that is positioned directly in front of the die 144 separating the die 144 from the optical fiber 141 (see FIG. 9C). The diameter of the lens 143 and refractive index of the material can be matched with the parameters described above to focus the widely diverging light from the emitter to the end of the optical fiber 141 thereby increasing the efficiency of transmission. FIG. 9A shows a flat bottom recess 142 and FIG. 9B shows a shaped bottom recess 142. In the case of the flat bottom recess 142, the flat polished end of the optical fiber 141 is mated with the bottom of the recess 142 using an optical adhesive. This creates a transparent junction between the LED lens material and the optical fiber 141. The geometry of the bottom of the recess 142 can be engineered to create a refractive surface in the optical path to help focus the light emitted from the die 144 onto the fiber 141. The geometry of the bottom of the recess 142 can be a simple spherical shape or an aspherical shape or an optimized optical surface. The adhesive/potting compound can be used that has a higher RI than the lens material on the die 144. When the optical adhesive is placed in the recess 142, it can fill the curved bottom of the recess 142 and create a focusing lens. The optical fiber 141 can have an outer diameter similar to the upper portion of recess 142 so that when the optical fiber 141 is potted it is not inserted past a point where the curved surface of the recess 142 begins. The higher RI of the optical adhesive lens focuses more light into the optical fiber 141.

The disposable portion can incorporate a light guide 102, an image guide 104, and a laser guide 106. Each of the guides can incorporate a single fiberoptic or a fiberoptic bundle. Where the term “fiberoptic” or “optical fiber” is used herein, both single fiberoptics and bundles of fiberoptics are considered. For example, where a single optical fiber is described as potted within a recess of the light source 122, multiple optical fibers or fiberoptic bundles can be potted.

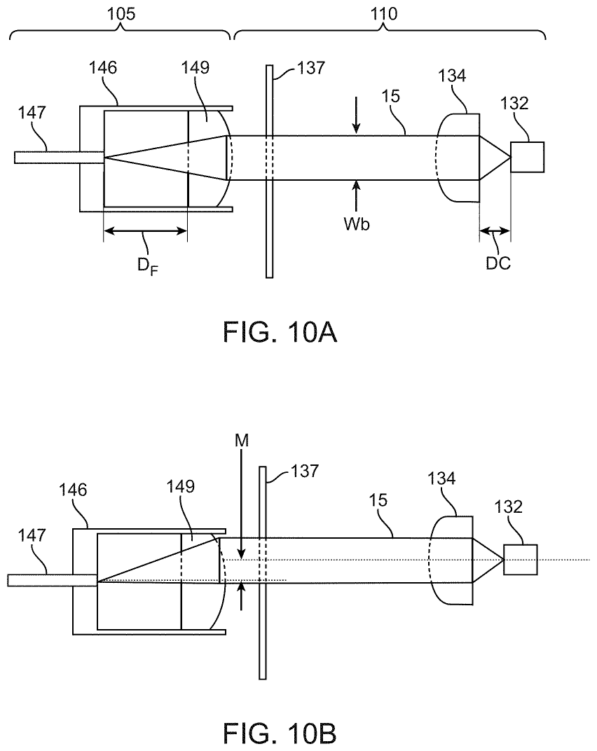

The laser guide 106 can include a single fiberoptic extending through the shaft 103 so that the distal end of the guide 106 is located at a distal end of the shaft 103 for treatments within the eye (see FIG. 5C). Upon coupling, the proximal laser guide 106 of the disposable portion 105 aligns with the laser treatment assembly 130 of the reusable portion 110. The laser treatment assembly 130 includes collimated optics that are directed towards the proximal end of the fiberoptic of the laser guide 106. FIGS. 10A-10B are schematics illustrating the optical chain for launching the laser beam 15 from the reusable portion 110 into the optical fiber 147 of the laser guide 106 of the disposable portion 105. The disposable portion 105 can incorporate optics that are configured to receive the laser beam 15 from the light tube 131 in a manner that focuses the light and injects the laser energy into the optical fiber 147 of the laser guide 106 of the disposable portion 105. The reversible mating between the two components—the disposable portion 105 and the reusable portion 110—may result in imprecise optical alignment between the laser beam from the light tube 131 of the reusable portion 110 and the laser guide 106 of the disposable portion 105. High tolerance axial alignment between the two components cannot always be guaranteed. To prevent lower efficiency injection of laser energy into the optical fiber 147 of the laser guide 106 when there is misalignment upon coupling of the two components of the device, an optical chain incorporating optics within the disposable portion 105 can be included. For example, a bushing 146 positioned within a proximal end region of the disposable portion 105 can affix an aspheric lens 149 between the proximal end of a fiber matched numerical aperture optical fiber 147 and the collimated beam of the laser penetrating the window 137 within the distal end of the reusable portion 110 (see also FIG. 4B). The optical fiber 147 can be about twice the size of the focusing spot size to allow for misalignment. For example, the fiber 147 can be about 200 microns in diameter and the focusing spot size of the beam can be only about 100 microns. The aspheric lens 149 creates an extremely small spot size from the collimated beam 15 for “injecting” the laser energy into the optical fiber 147. The focusing lens 149 can have a numeric aperture about 0.1-0.5, preferably between 0.2-0.3. The focusing lens 149 can have a focal length that is about 5-20 mm, about 4-12 mm, preferably about 7-9 mm. The focusing lens 149 can have a lens diameter that is about 3-20 mm, preferably about 4-5 mm. The focusing lens 149 can have an asphericity that is optimized for a single focal point diameter of 3-8 mm, for example, about 3-6 mm. The laser optical fiber numerical aperture can be about 0.1-0.7, preferably about 0.2-0.3. The laser optical fiber can have a core diameter that is about 10-1000 preferably about 100-300 μm.

FIG. 10A shows the optical chain with high tolerance axial alignment between the laser beam 15 and the optical fiber 147. FIG. 10B shows the optical chain where the axial alignment between the two is slightly off-set. The collimated beam 15 from the reusable portion 110 is focused by the aspheric focusing lens 149 within the disposable portion 105. The focusing lens 149 is positioned a focusing distance Df away from the proximal end of the optical fiber 147. The beam 15 from the laser assembly hits the lens 149 in the clear aperture area of the focusing lens 149, which ensures that even if the marginal rays of the collimated laser beam 15 hit the edge of the clear aperture of the focusing lens 149, the beam 15 will still come to focus on the end of the optical fiber 147. The tolerance for misalignment is a function of the collimated beam width W_{b} relative to the aspheric lens 149 clear aperture. The smaller the beam width W_{b} relative to the clear aperture, the more misaligned the optical path can be. FIG. 10B shows a high numeric aperture collimating lens 134 in the reusable portion 110 of the device positioned close to the laser diode 132 separated by a collimating distance D_{c} and resulting in a smaller beam width W_{b} (compared to a collimating lens with a longer focal length) and having a misalignment M. The closer the collimating lens 134 is positioned to the laser diode emitter 132, the less distance for beam divergence prior to hitting the collimating lens 134. The short distance between the lens to the emitter demands a high numeric aperture and a short focal length collimating lens 134. The distance can be about 2-3 cm. The small collimated beam width W_{b} allows full beam to enter the focusing lens 149 of the disposable portion 105 even with axial misalignment. An appropriately designed aspheric focusing lens 149 corrects for spherical aberration and provides perfect focus of both paraxial and marginal rays so that all the light enters the optical fiber end 147.

FIGS. 2A-2C, FIG. 4B, and FIGS. 10A-10B each show a laser treatment assembly 130 having a laser diode emitter 132 that is positioned within the reusable portion 110. The light crosses the junction between the housing portions 112a, 112b to the aspherical lens 149 within the disposable portion 105 in order to direct laser energy for a treatment at the distal end of the shaft 103. The laser treatment assembly 130 may also be located fully within the disposable portion 105. FIG. 11 shows an implementation of the laser treatment assembly 130 that is located fully within the disposable portion. The laser treatment assembly 130 can include a laser diode 132 and a ball lens 156 positioned between the laser diode emitter 132 and the optical fiber 147. The laser diode emitter 132 (e.g., 808 nm) includes a base 160 coupled to an annular casing 161 having an inner diameter defining an aperture 165 extending through the casing. A glass window 162 is mounted within the casing 161 forming a recess 163 on an end of the casing 161. The recess 163 is located opposite the base 160 of the laser diode emitter 132 separated by the glass window 162. The ball lens 156 sits within the recess 163 of the casing 161 and can be held in place against the casing 161 by a housing 158 surrounding the casing 161 and the ball lens 156. The ball lens 156 is retained within the recess 163 in appropriate alignment.

The ball lens 156 can have a diameter, index of refraction, and material selected to account for specific laser diode emitter 132 divergence characteristics. The ball lens 156 can be designed to have an outer diameter sized to be received only partially within the recess 163 such that the annular casing 161 supports the ball lens 156. The size of the recess 163 can be slightly smaller than the outer diameter of the ball lens 156 so that the ball lens 156 can be supported by the inner diameter of the laser diode emitter 132 recess 163. For example, the laser diode emitter 132 of the laser treatment assembly 130 can be about 5 mm in diameter and incorporate a recess 163 of about 1 mm in diameter. The ball lens 156 can be about 1.5 mm in diameter so as to sit partially within, but remain substantially outside the recess 163 so that a majority of the ball lens 156 is external to the recess 163.

The laser diode emitter 132 emits in a rapidly diverging ellipsoid having an emitting point 164. The ball lens 156 resting within the recess 163 of the laser diode emitter 132 casing 161 focuses the laser light into the optical fiber 147 proximal end. The optical fiber 147 can be attached to the ball lens 156 such as by gluing or can be suspended a tiny fraction away from the ball lens 156 such as with a fiber ferule 159. The optical fiber 147 can be held by the fiber ferule 159 to be positioned normal to the light path. The distance between the optical fiber 147 and the ball lens 156 can be adjusted to set the optimal focus. The optical fiber 147 can be aligned with the brightest image transmitted through the lens 156. This allows for the reusable portion 110 to incorporate only the imaging assembly 125 while the illumination assembly 120 and laser treatment assembly 130 are positioned within the disposable portion 105 and need not be autoclaved. Incorporating the laser treatment assembly 130 fully within the disposable portion 105 means the beam does not need to cross the junction between the reusable 110 and disposable 105 portions solving any alignment issues. The laser treatment assembly 130 incorporating fewer components and is less expensive. Perfect focus can be achieved with one adjustment (i.e., the ferule-in-shaft position) making the set focus easier during production.

The fiber 147 can have a numeric aperture that is about 0.1-0.6, preferably about 0.20-0.25. The recess diameter can be about 800-2000 microns, preferably about 1000-1700 microns. The ball lens 156 diameter can be about 1000-3000 microns, preferably about 1500-2500 microns and greater than the recess diameter. The ball lens refractive index can be about 1.45-2.0, preferably about 1.75-1.80. The distance between the ball lens 156 and the optical fiber 147 can be from 0-10 mm, preferably about 250-400 microns. The material of the ball lens as well as any of the lenses described herein can vary. Example materials of the lenses are BK7, fused silica, magnesium fluoride, zirconia, sapphire, ruby, etc, or any optical material including optical polymers. Sapphire, in particular, is a suitable material for any of the lenses described herein due to its ability to withstand heating to high temperatures without melting and cooling again. Sapphire also has a very high refractive index that is able to bend light well.

Power levels of the 810 nm laser to achieve whitening and shrinkage of the ciliary tissue in the eye can be between 100-300 mW, typically about 250 mW at the treatment site. Conventional systems may have lasers that use anywhere up to about 1.0-2.0 W of power recorded at the console, but there is a loss in power within the eye due to lower efficiency (e.g., about 50% loss to about 500 mW). The efficiency in the transmission of laser energy between the reusable and disposable portion of the devices described herein is very high (e.g., about 25% loss) such that the power needs of the laser is reduced.

The disposable portion 105 can connect via a fluid port (not shown) to a fluid channel such as tubing from an IV bag to supply liquid to the distal shaft during use. The delivery of the liquid can be passive gravity-assisted fluid delivery or can be assisted by a pump as is known in the art. The liquid being delivered can be an irrigation liquid such as saline. The liquid can be cooled to aid in cooling during a procedure with the laser. The tubing to deliver the liquid to the shaft 103 can be positioned relative to the shaft 103 such as with an irrigation sleeve surrounding at least a portion of the shaft 103. Even when the irrigation sleeve is present, the maximum cross-sectional diameter of the shaft 103 is preferably suitable for minimally-invasive procedures in the eye to minimize the incision size. The maximum cross-sectional diameter of the probe is preferably about 1.25 mm. The maximum cross-sectional diameter can be smaller than this or can be larger than this diameter, for example, no more than about 2 mm in diameter, no more than about 3 mm in diameter, up to about 4 mm in diameter, or up to about 5 mm in diameter. The fluid can be transported through either tubing adjacent to the shaft as described above or through the inside of the shaft 103. For example, the shaft 103 can incorporate a microfluid channel in addition to the light guides. The fluid transported by the tubing or channel can vary depending on the needs of the procedure being performed. For example, glaucoma can be treated using the endoscopic device by delivering cold water ab interno through the elongate shaft while applying laser energy to a ciliary body of the eye through the elongate shaft. The fluid can be irrigation liquid such as saline, cold liquids for cooling such as water or saline, liquids for inflammation reduction, anti-inflammatory therapeutic formulations, antibiotic formulations, or other therapeutic formulations that are clinically relevant for the procedure being performed. Various dyes, medicaments, and liquids are delivered during ophthalmic surgeries including for glaucoma treatments, vitrectomy, cataract surgery, and macular surgeries. The liquids can include therapeutic dyes such as Trypan blue, indocyanine green (ICG), Lissamine green, Rose Bengal, Triamcinolone Acetonide, Brilliant Blue G/Acid Blue, light green SF, bromophenol blue, Patent Blue, Chicago blue, E68, Fluorescein,

The disposable portion 105 and/or the reusable portion 110 can include a basic user interface 115 including actuators such as buttons, wheels, switches, sliders, dial, switch, pedal, and other actuator configured to operate one or more aspects of the device 100. The implementation shown in FIGS. 4A-4B, 4C-4D, and FIGS. 5A-5B includes an actuator 115 on the disposable portion 105 that is configured to provide multi-way triggering of the device 100 such as using a Hall sensor or other mechanism. The actuator 115 can be moved by a user from a home position where all functions of the device 100 are off to at least a first position to turn on a function of the device 100 such as the treatment laser. Further movement of the actuator 115 away from the home position can increase the intensity of the laser power. The actuator 115 is shown as a slider, but the actuator 115 can also be a trigger that is configured to be depressed a number of degrees to increase the intensity. The actuator 115 can move between set positions to achieve pre-programmed intensity positions or can move along a variety of infinite positions between home and full intensity. The device 100 can have different actuator(s) 115 to power on one or more other functions of the device 100 such as an actuator that turn on one or both of the illumination light source 122 and the imaging assembly 125. As an another example, the device 100 can have actuators to control LEDs of different wavelengths such as one actuator 115 for a white light LED for visible viewing and another actuator 115 for near-UV fluorescent viewing and another actuator 115 for photobiomodulation LEDs (e.g., 808 nm LED light). In still further implementations, the device 100 can incorporate a remote control box to free up space on the handle in order to control various functions. Alternatively, the illumination light source 122 and imaging assembly 125 may be powered on upon connecting the device 100 to a power source.

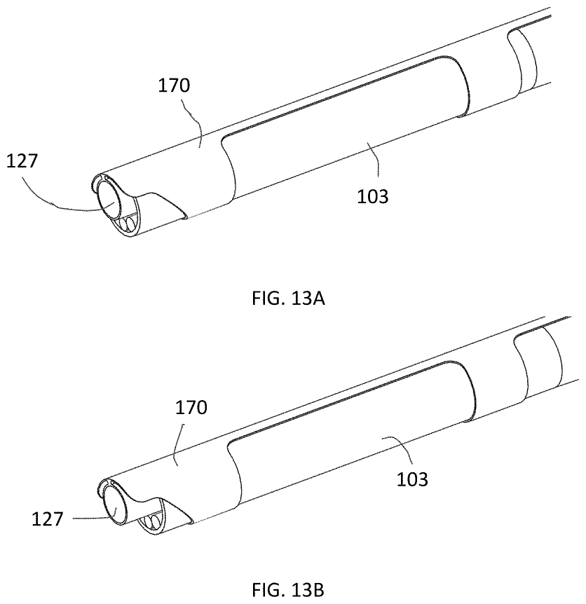

The device need not include a mechanism to change optical focus point of the imaging assembly 125. The device can include a focus mechanism that allows for manual or automatic focusing adjustment of the depth of field (e.g., 1-30 mm). The depth of focus may be adjusted by moving the fiber bundle 104 relative to the objective lens 127 or move the lens 127 relative to the fiber bundle 104. The device 100 can incorporate a separate actuator 115 for changing the position of one or both of the fiber optic bundle 104 and objective lens 127. The movement achieved by the actuator can be about 200-250 microns. The movement can be achieved using a micro piezo actuator or another linear actuator. FIGS. 13A-13B illustrate an example of a system configured to vary the distance between the objective lens 127 and the fiber optic bundle 104. The probe shaft 103 can incorporate an outer sleeve 170 having a proximal end and a distal end. The distal end of the sleeve 170 can be coupled to the objective lens 127 so that the sleeve 170 remains outside the shaft 103 and the lens 127 coupled to the sleeve 170 is positioned inside the shaft 103. The proximal end of the sleeve 170 can be coupled to a linear actuator such as a piezo actuator configured to move the sleeve 170 to vary the focal distance of the objective lens 127. FIG. 13A illustrates the sleeve 170 in the proximal position retracted so as to cause the objective lens 127 to be positioned close to the distal end of the fiber optic bundle 104 (not visible in FIG. 13A). FIG. 13B illustrates the sleeve 170 in the distal position extended so as to cause the objective lens 127 to move away from the distal end of the fiber optic bundle 104. The sleeve 170 can be fully tubular or can be only partially tubular as shown in FIGS. 13A-13B having a pair of straps configured to surround the shaft 103.

The device can also incorporate user feedback or outputs 117 such as visual, auditory, and/or tactile outputs. The output 117 can include one or more of a light, display, speaker, vibration motor, or other sort of output configured to communicate information to the user by visual, audio, and/or tactile outputs. As an example, the disposable portion 105 can include a light (e.g., LED) that illuminates a light pipe that is visible from outside the housing 112a. The LED can blink when the device 100 is powered on, but before the laser is activated. Once the laser is activated the LED can remain steadily lit up to let the user know the laser is on. The LED(s) can be any of a variety of colors. For example, rather than changing from a blinking light to a steady light, the LED can change from a green light to a red light to let a user know the laser is active. The position of the outputs 117 can vary depending on the arrangement of the housing 112, but are located so that a user can readily observe them during use. It should also be appreciated that one or more inputs and outputs can be located on the disposable portion, the reusable portion 110, or both.

Methods of Use

As an example method of use, the eye can be penetrated via a limbal or pars plana approach to access the ciliary process. Access of the ciliary process can be achieved from an anterior approach in phakic, pseudophakic, aphakic eyes. If the photocoagulation procedure is planned in combination with cataract surgery and IOL implantation, a limbal approach is preferred. Alternatively, a scleral tunnel incision can used in cataract surgery to provide access for the device. Pars plana approach can be used in pseudophakics and provides a good view of ciliary processes. Anterior vitrectomy may be performed when using this approach.

An incision (e.g., 1.5-2.2 mm long) may be created using a cutting tool for clear corneal incisions or a puncture tool and the sulcus can be deepened using ophthalmic viscoelastic. The probe on the disposable portion coupled to the reusable portion can be connected via the cable with a video monitor providing real-time imaging and power. Video imaging can be connected to monitor via wireless connection. Laser energy is delivered to whiten the ciliary process and produce visible tissue shrinkage. Photocoagulation can be applied a distance away from the ciliary processes, such as about 1.0 mm-3.0 mm from ciliary processes, preferably about 2 mm. Slow, continuous “painting” of each ciliary process along at least about 270 degrees up to about 360 degrees (through one incision with a curved endoscopic probe or through two incisions) can be performed.

There are various ways to approach the ciliary processes and many techniques that can be employed depending on lens status, type and severity of the disease being treated. Endoscopic photocoagulation (ECP) approaches and surgical techniques can vary including limbal/clear corneal, pars plana, over-the-bag, through-the-bag, phakic eye anterior approach, aphakic eye, pseudophakic eye, ECP plus for refractory glaucoma, endoscopic cycloplasty (ECPL), endocilioplasty for plateau iris syndrome, ICE procedure (MIGS implant combined with cataract surgery and ECP, cataract, ECP, ECP in keratoprosthesis glaucoma treatment, uveitis glaucoma hyphema syndrome, pseudoexfoliation glaucoma, ECP for cyclodialysis cleft.

The endoscopy devices described herein permit imaging, illumination, and laser application when anterior or posterior conditions preclude a posterior view. Conditions that can be treated include opaque cornea, opaque anterior chamber, small pupil, opacified lens, disrupted lens implant, gas in the vitreous, and others. The endoscopy devices described herein allow viewing anatomy or pathology that is not visible through operating microscopes including posterior iris, ciliary body, pars plana, peripheral retina, intraocular foreign body, dislocated lens implants, and angle for goniotomy.

The endoscopy devices described herein assist in unique surgical techniques including incarcerated vitreous in sclerotomies, origin enigmatic intraocular hemorrhage or infection, subretinal placement of infusion cannula, sclerotomy induced retinal detachment, vitreous not observed through microscope, creation of posterior vitreous separation in macular hole surgery. The endoscopy devices described herein allow for endoscopically monitoring vitrectomy, membranectomy, bloc dissection, air-, liquid-, or silicone oil-fluid exchange, internal drainage of subretinal fluid, retinotomy/retinectomy, lensectomy, retinal detachment—laser endoscopic repair. Disease-specific applications considered herein include surgical management of diabetic retinopathy and its complications, retinal detachment, proliferative vitreoretinopathy, dislocated cataract, dislocated lens implant, neovascular glaucoma, endophthalmitis, intraocular foreign body, hypotony, and choroidal hemorrhage.

The endoscopy devices described herein need not be limited to usage in the eye. For example, the endoscopy device can be used in ENT sinus surgery, for example, to shrink polyps. The probe shaft length can be changed depending on intended anatomy. A probe shaft may be increased for use in sinus surgeries to treat polyps or chronic sinus inflammation and depending on which sinus it being treated (e.g., maxillary sinus).

The system can include a control unit, power source, microprocessor computer, and the like. Aspects of the subject matter described herein may be realized in digital electronic circuitry, integrated circuitry, specially designed ASICs (application specific integrated circuits), computer hardware, firmware, software, and/or combinations thereof. These various implementations may include an implementation in one or more computer programs that are executable and/or interpretable on a programmable system including at least one programmable processor, which may be special or general purpose, coupled to receive signals, data and instructions from, and to transmit signals, data, and instructions to, a storage system, at least one input device, and at least one output device.

These computer programs (also known as programs, software, software applications, or code) include machine instructions for a programmable processor, and may be implemented in a high-level procedural and/or object-oriented programming language, and/or in assembly/machine language. As used herein, the term “machine-readable medium” refers to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the devices to a specific configuration described in the various implementations.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value. In aspects, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The systems disclosed herein may be packaged together in a single package. The finished package would be sterilized using sterilization methods such as Ethylene oxide or radiation and labeled and boxed. Instructions for use may also be provided in-box or through an internet link printed on the label.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claims. No language in the specification should be construed as indicating any non-claimed element essential to the practice of the invention.

Groupings of alternative elements, embodiments, or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

### P Embodiments

P Embodiment 1. An intraocular—handheld cyclophotocoagulation device having a reusable handle body containing a light source, imaging unit, laser diode, wireless video transmitter, and focal length adjustment and a disposable endoscopic distal tip containing a sheath, a light guide/lens, a laser guide/lens, and an image guide/lens.

P Embodiment 2. The device of P Embodiment 1, where a multiple, single, or grouped lumen contained within a distal tip and shaft provide visible light to and from the designated therapy area is intended to be a single use disposable.

P Embodiment 3. The device of P Embodiment 1, where the distal shaft has an image transfer lumen having field of view of at least between 45 and 150 degrees.

P Embodiment 4. The device of P Embodiment 1, where a focus mechanism of the reusable portion has a manual focusing adjustment allowing 1 to 30 mm depth of field.

P Embodiment 5. The device of P Embodiment 1, where a focus mechanism of the reusable portion has an automatic focusing adjustment allowing 1 to 30 mm depth of field.

P Embodiment 6. The device of P Embodiment 1, where the distal shaft has a single, multiple single, or grouped lumens to transmit 810 nm wavelength light from laser diode to the designated therapy area and is intended to be a single use disposable.

P Embodiment 7. The device of P Embodiment 1, where the distal tip is coupled to the reusable handheld portion of the device to minimize losses.

P Embodiment 8. The device of P Embodiment 1, where the reusable handle body houses control electronics and illumination sources and is intended to be reusable.

P Embodiment 9. The device of P Embodiment 1, where the reusable handle body is able to wirelessly transmit video and data to a screen, computer, or other handheld device.

P Embodiment 10. The device of P Embodiment 1, wherein the reusable handle body contains LED transmitters that include white, blue, or green visible light for the purpose of photobiomodulation.

1. An intraocular cyclophotocoagulation device comprising:
a proximal, reusable portion comprising:
a proximal housing portion having a distal end region;
a laser treatment assembly comprising a laser diode and a collimating lens positioned a distance distal to the laser diode and configured to collimate light from the laser diode into a collimated laser beam and direct the collimated laser beam towards the distal end region of the proximal housing portion; and
an imaging assembly; and
a distal, disposable portion comprising:
a distal housing portion having an elongate shaft extending distally from a distal end region of the distal housing portion, the distal housing portion having a proximal end region configured to reversibly couple with the distal end region of the proximal housing portion;
a laser guide extending through the elongate shaft and an aspheric lens positioned within the distal housing portion to receive the collimated laser beam from the proximal reusable portion and direct the collimated laser beam toward a proximal end of a fiberoptic of the laser guide;
an imaging guide extending through the elongate shaft and an objective lens located at a distal end region of the elongate shaft, the objective lens arranged to focus an image onto a distal end of a fiberoptic of the imaging guide; and
an illumination light guide extending through the elongate shaft, an illumination source positioned within the distal housing portion and arranged relative to a proximal end of a fiberoptic of the illumination light guide.

2. The device of claim 1, wherein the laser diode of the laser treatment assembly transmits in a near-infrared wavelength that is configured to burn and shrink tissue.

3. The device of claim 1, wherein the collimated laser beam is unchanged with minimal divergence or convergence crossing a junction from the reusable portion to the disposable portion.

4. The device of claim 1, wherein the aspheric lens corrects for spherical aberration and provide focus of both paraxial and marginal rays so that all light of the collimated laser beam enters the proximal end of the fiberoptic of the laser guide.

5. The device of claim 1, wherein the fiberoptic of the laser guide is about 200 microns in diameter and the collimated laser beam has a focusing spot that is about 100 microns.

6. The device of claim 1, wherein the objective lens is monolithic and apertureless.

7. The device of claim 1, wherein the objective lens has a frustoconical configuration having a smaller diameter entry surface and a maximal diameter exit surface, the exit surface positioned proximal to the entry surface near the distal end of the fiberoptic of the imaging guide.

8. The device of claim 1, wherein the objective lens has an hourglass configuration comprising an entry surface, an exit surface and a neck located between the entry surface and the exit surface, wherein the neck has a smaller diameter than a diameter of the entry surface or a diameter of the exit surface.

9. The device of claim 1, wherein the objective lens has a depth of focus that is between 1-6 mm.

10. The device of claim 1, wherein the proximal end of the fiberoptic of the illumination light guide is affixed on or near an emitting die of the illumination source.

11. The device of claim 10, wherein the fiberoptic of the illumination light guide is potted in a recess of the illumination source.

12. The device of claim 11, wherein the recess has a curved bottom and together with an optical adhesive in the recess creates a focusing lens between the fiberoptic of the illumination light guide and the emitting die of the illumination source.

13. The device of claim 1, further comprising an actuator that is a slider configured to increase intensity of the laser light emitted.

14. The device of claim 13, wherein the slider is positioned on the disposable portion.

15. The device of claim 1, wherein the elongate shaft is curved.

16. The device of claim 15, wherein the device provides a field of view that is between 45-150 degrees.

17. The device of claim 1, wherein the distal end region of the proximal housing portion tapers and is sized to be received within a corresponding shaped region at the proximal end region of the distal housing portion.

18. The device of claim 17, wherein the distal end region of the proximal housing portion and the proximal end region of the distal housing portion couple together under a spring-load.

19. The device of claim 1, further comprising a fluid channel extending within the disposable portion.

20. The device of claim 19, wherein the fluid channel extends through the elongate shaft.

21. The device of claim 19, wherein the fluid channel is configured to deliver cooled liquid to an eye.

22.-67. (canceled)

68. An endoscopic device for intraocular treatment, the device comprising:
a proximal, reusable portion comprising a proximal housing portion having a distal end region; and
a distal, disposable portion comprising a distal housing portion having an elongate shaft extending distally from a distal end region of the distal housing portion, the distal housing portion having a proximal end region configured to reversibly couple with the distal end region of the proximal housing portion, a frustoconical lens positioned within a distal end region of the elongate shaft.

69. The device of claim 68, further comprising an imaging channel extending within the elongate shaft configured to transmit light from the frustoconical lens.

70. The device of claim 69, further comprising one or both of an illumination channel and a laser treatment channel extending within the elongate shaft.

71. A method of treating glaucoma using an endoscopic device comprising an elongate shaft, the method comprising delivering cold water ab interno through the elongate shaft while applying laser energy to a ciliary body of an eye through the elongate shaft.
